(19) United States
(12) Patent Application Publication
(10) Pub. No.: US 2010/0009373 A1
(43) Pub. Date: Jan. 14, 2010

(54) METHODS AND COMPOSITIONS RELATING TO MULTIPLEX GENOMIC GAIN AND LOSS ASSAYS

(75) Inventors: Randall Walker, Framingham, MA (US); Karl Edwin Adler, Newburyport, MA (US)

Correspondence Address: GIFFORD, KRASS, SPRINKLE, ANDERSON;& CITKOWSKI, P.C., P.O. BOX 7021, TROY, MI, 48007-7021, US

(73) Assignee: PerkinElmer Health Sciences, Inc., Waltham, MA (US)

(21) Appl. No.: 12/512,680

(22) Filed: Jul. 30, 2009

Related U.S. Application Data
(63) Continuation-in-part of application No. 12/275,895, filed on Nov. 21, 2008.
Application No. 12/275,895 is a continuation-in-part of application No. 11/615,739, filed on Dec. 22, 2006.
Application No. 12/275,895 is a continuation-in-part of application No. 12/055,919, filed on Mar. 26, 2008.
(60) Provisional application No. 60/753,584, filed on Dec. 23, 2005.
(60) Provisional application No. 60/753,822, filed on Dec. 23, 2005.
(60) Provisional application No. 60/765,311, filed on Feb. 3, 2006.
(60) Provisional application No. 60/765,355, filed on Feb. 3, 2006.
(60) Provisional application No. 60/992,489, filed on Dec. 5, 2007.

(51) Int. Cl. C12Q 1/68

(52) U.S. Cl. 435/6

(57) ABSTRACT

Compositions and methods are provided for detecting genomic DNA gain and loss. Embodiments of inventive compositions and methods include composite nucleic acid probes which specifically hybridize to two or more genomic loci in a genomic region of a reference genome for detection of genomic gain and/or loss in a subject genome. In some embodiments, a substrate-attached composite nucleic acid probe is provided which includes a mixture of separate populations of beads having attached DNA probes wherein all of the beads are identically encoded and wherein each individual bead has exclusively DNA derived from one source, such as a particular large insert vector containing chromosomal DNA, or amplicons generated by amplification of DNA derived from a large insert vector containing chromosomal DNA.

### REFERENCE TO RELATED APPLICATIONS

This application is a continuation-in-part of U.S. patent application Ser. No. 12/275,895, filed Nov. 28, 2008, which is a continuation-in-part of U.S. patent application Ser. No. 12/055,919, filed Mar. 26, 2008, which claims priority from U.S. Provisional Patent Application Ser. No. 60/992,489, filed Dec. 5, 2007.

U.S. patent application Ser. No. 12/275,895 is also a continuation-in-part of U.S. patent application Ser. No. 11/615,739, filed Dec. 22, 2006, which claims priority from U.S. Provisional Patent Application Ser. Nos. 60/753,584, filed Dec. 23, 2005; 60/753,822, filed Dec. 23, 2005; 60/765,311, filed Feb. 3, 2006; and 60/765,355, filed Feb. 3, 2006. The entire content of each application is incorporated herein by reference.

### FIELD OF THE INVENTION

Technology described herein relates generally to methods and compositions for detection of nucleic acids. More specifically described are methods and compositions for genomic gain and loss assays.

### BACKGROUND OF THE INVENTION

Assays for detection of genomic gain and loss allow for detection and diagnosis of genetic abnormalities which can underlie disease, behavioral and cognitive conditions, and other genetic-based pathologies.

Array-CGH (aCGH) is a multiplex assay method where immobilized DNA probes capture labeled complementary genomic DNA sequences from a test sample and a reference sample. Each probe generates a ratio of the test/reference amounts of one of the constituent sequences defined by the probe sequence. In aCGH it is common to use bacterial artificial chromosome (BAC) DNA as the probe material immobilized onto the array's solid support. Typical BAC sequence length is between about 100 kilobases and about 250 kilobases, with 175 kilobases length typical. Probes of this length are well-suited to aCGH in that they are long enough to generate large hybridization signals and to not respond to small mutations such as SNPs, yet short enough to provide sufficient genomic resolution to detect the break points of a genomic gain or loss with some precision. For example, a well-designed BAC array can detect different sizes of deletion regions in microdeletion syndromes such as 1p36 deletion syndrome or DiGeorge syndrome, where deletions spanning different portions of the region associated with the disorder may correspond to different phenotypes.

The trend in the design of so-called constitutional aCGH microarrays has been toward using multiple separate probes, such as BAC probes in each of the genomic regions associated with inborn DNA disorders to detect the extent of genomic gains and losses with higher resolution. The higher resolution reveals more information on the exact size and boundaries of a gain or loss region. However, there are cases in which high resolution detection of the exact size and boundaries of a gain or loss region may not be necessary.

Aneuploidies are examples of genomic gain-loss anomalies where detecting breakpoints at high resolution is not relevant. Aneuploidies are a gain (most typically, e.g. trisomy) or a loss of an entire chromosome (average size over 100 megabases), as opposed to a microdeletion disorder where the deleted region may be only from typically 1 to 10 or so megabases in extent. Detection of aneuploidy (also known as chromosome enumeration) can be done with as few as one BAC probe in an aCGH assay, however noise on the sample/normal ratio from that single probe can lead to ambiguity about the result. The use of multiple probes targeted to the subject chromosome where the majority of the probes all show the same gain or loss provides more confidence in an aneuploidy detection result from an aCGH assay, and that is the routine construction of CGH arrays. Most array analyses require concordant ratio signals from at least two or three or more BAC probes in a region before a gain or loss is definitively “called” or determined.

Additional individual probes in the gain or loss region increases the confidence of the detection of the gain or loss which is valuable in cases where the assay signal ratios are noisy. Noise is present in assays for a variety of reasons. Sometimes the assay is simply noisy due to non-uniform conditions of either probe immobilization or sample incubation or due to non-optimum conditions in labeling, hybridization, washing, or drying. In other cases, noise may be induced by the sample having been amplified using whole-genome amplification (WGA) such as the phi-29 or DOP PCR methods, where the amplified sample shows sequence-specific amplification bias. WGA is used when the initial sample amount is limited, such as the DNA from a single cell or from just a few cells. Such amplified samples have varying DNA product yields in different parts of the genome (amplification bias), superimposing genomic gain-loss noise onto the assay that generally results in significant variation of sample-to-reference ratio response between multiple individually arrayed or assayed BAC probes. The standard method for compensating for this noise is to use a plurality of probes to span the genomic sequence region of interest and in some manner, such as averaging the ratio responses across the genomic region, utilizing the composite results of the plurality of probes to make a gain-loss call across the region. Averaging of ratios across multiple individually arrayed or assayed probes is particularly common in oligonucleotide CGH arrays, which typically show more probe-to-probe ratio variation than BAC arrays.

Unfortunately, use of multiple individually arrayed or assayed probes directed to the same region in order to increase confidence can be limiting. For example, there are configurations of immobilized probe arrays where the use of multiple individually arrayed or assayed probes to detect each aneuploidy is problematic. Some array CGH formats are restricted in the number of probes that they can accommodate. An example of such an array is one printed on the bottom of a microplate well, or one printed on just a small segment of a microscope slide substrate that is configured to accommodate multiple separate samples on a single substrate. These arrays commonly utilize between 9 (3×3 spot array matrix) and 100 (10×10) probes. In such a 100-plex case a maximum of 4 probes could be accommodated for enumerating each of the 24 chromosomes (1-22, X and Y), and effectively the entire capacity of the array would have been used up for chromosome enumeration.

Besides planar microarrays another method for measuring genomic gains and losses in a sample using multiple probes simultaneously is the use of a set of encoded particles or microspheres with immobilized probes. The most widely used encoded particle platform is the Luminex xMAP system that uses fluorescent color-coding to distinguish 100 different microsphere or bead types. This system can support a maximum of 100 different probes in a genomic gain-loss assay. This encoded microsphere platform does not support the two-dye two-color readout of ratio results from competitive hybridization as microarrays do, but the same ratios can be generated by separate side-by-side assays of test and reference samples, normalizing appropriately, and calculating the ratios.

In these multiplex assay formats where the maximum numbers of probes is limited there is a continuing need to reliably assay for aneuploidies of up to 24 chromosomes (1 through 22, plus X and Y) without utilizing the entire or even the majority of the possible set of probes just for that purpose. For example, in constitutional disorder assay panels it is most often desired to probe for several microdeletion disorders in addition to aneuploidies. Also, assaying for microdeletion disorders (such as Wolf-Hirschlhorn, Willams-Beuren, Cri-du-Chat and the like) can be done robustly using one or two lower resolution probes rather than a larger number of conventional probes, allowing more disorders to be assayed on a limited-probe platform Similarly in cancer cytogenetics there are generally several regions where fairly high-resolution gain-loss data is required in addition to other areas where the loss of an entire chromosome or chromosome arm is the desired resolution. Such panels would be more useful if the genomic regions requiring only low resolution did not utilize more than a few probes in the panel, freeing up assay platform capacity for more probes for the regions needing higher resolution.

Therefore, methods and compositions are required for reliably assaying for chromosome-scale and chromosome-arm scale gains and losses while allowing for the option of assaying other genomic regions simultaneously with one or more higher resolution probes.

### SUMMARY OF THE INVENTION

A method of assaying a DNA sample is described which includes providing a substrate-attached composite nucleic acid probe. The composite nucleic acid probe includes nucleic acid sequences which specifically hybridize to two or more genomic loci in a genomic region of a reference genome. The genomic region is characterized by a first terminus and a second terminus with an intermediate region of at least 400 kilobases disposed between the termini. The composite nucleic acid probe includes nucleic acid sequences which specifically hybridize to substantially an entire first genomic locus which includes the first terminus of the genomic region and nucleic acid sequences which specifically hybridize to substantially an entire second genomic locus which includes the second terminus. The first genomic locus and second genomic locus each typically include at least about 100 kilobases. Optionally, nucleic acid sequences of the composite probe specifically hybridize to additional loci within the genomic region.

The substrate-attached composite nucleic acid probe is hybridized with sample genomic DNA at a stringency sufficient to achieve specific hybridization. The substrate-attached composite nucleic acid probe is also hybridized with reference genomic DNA.

A first signal indicating specific hybridization of the substrate-attached composite nucleic acid probe with the sample genomic DNA is detected along with a second signal indicating specific hybridization of the substrate-attached composite nucleic acid probe with the reference genomic DNA. The first signal and the second signal are compared to detect differences between the first and second signals, indicative of differences between the sample DNA and the reference DNA.

The nucleic acid sequences which specifically hybridize to two or more genomic loci in a genomic region of a reference genome can be derived from two or more large-insert DNA vectors. For instance, the nucleic acid sequences are derived from two or more large-insert DNA vectors such as bacterial artificial chromosomes, yeast artificial chromosomes human artificial chromosomes, cosmids, plasmids, phagemids, phage DNA and fosmids. The nucleic acid sequences which specifically hybridize to two or more genomic loci in a genomic region of a reference genome can also be derived from isolated chromosomes and/or isolated chromosome fragments.

In a particular option, the nucleic acid sequences which specifically hybridize to two or more genomic loci in a genomic region of a reference genome are amplicons derived from two or more large-insert DNA vectors, isolated chromosomes, isolated chromosome fragments or a combination of these or other sources of nucleic acids.

In a particular option, the substrate is a plurality of particles, such as a plurality of encoded particles. In a further option, the substrate is a planar substrate.

The nucleic acid sequences which specifically hybridize to two or more genomic loci in a genomic region of a reference genome individually have a length in the range of about 20-250,000 nucleotides, inclusive.

A method of assaying sample nucleic acid is provided which includes providing a multiplex reagent including a mixture of two or more encoded particle sets encoded such that each particle of each encoded particle set is detectably distinguishable from each particle of each other encoded particle set. The encoded particles include attached nucleic acid sequences which specifically hybridize to at least one genomic locus of a reference genome, and at least one encoded particle set includes an attached composite nucleic acid probe.

The multiplex reagent is hybridized with sample genomic nucleic acid and with reference nucleic acid, together or in parallel.

A first signal is detected which indicates specific hybridization of the attached nucleic acid sequences with detectably labeled sample nucleic acid and a second signal is detected indicating specific hybridization of the attached nucleic acid sequences with detectably labeled reference nucleic acid. The encoded particles are then identified so as to associate particle encoding with the first signal or with the second signal. The first signal and the second signal for each encoded particle set are then compared and differences in the first and second signals are indicative of differences between the sample and reference nucleic acids.

A reagent for assay of nucleic acids is provided which includes a first composite nucleic acid probe attached to a solid substrate. The first composite nucleic acid probe includes nucleic acid sequences which specifically hybridize to two or more genomic loci in a genomic region of a reference genome. The genomic region is characterized by a first terminus and a second terminus and has an intermediate region disposed between the first terminus and second terminus of at least 400 kilobases. The first composite nucleic acid probe includes nucleic acid sequences which specifically hybridize to substantially an entire first genomic locus including the first terminus of the genomic region and nucleic acid sequences which specifically hybridize to substantially an entire second genomic locus including the second terminus of the genomic region.

In a particular option, a reagent for assay of nucleic acids includes at least two and may include more composite probes. For example, a second composite nucleic acid probe attached to a solid substrate includes nucleic acid sequences which specifically hybridize to two or more genomic loci in a second genomic region of a reference genome. The second genomic region is characterized by a first terminus and a second terminus and has an intermediate region disposed between the first terminus and second terminus of at least 400 kilobases. The second composite nucleic acid probe includes nucleic acid sequences which specifically hybridize to substantially an entire first genomic locus comprising the first terminus of the second genomic region and to substantially an entire second genomic locus comprising the second terminus of the second genomic region.

The solid substrate is a planar substrate in one option. An array including one or more composite probes can be included on a planar substrate. In a further option, non-composite probes can also be included on the planar substrate to provide a panel of composite and other probes.

In a further aspect, the solid substrate is a first plurality of particles. A second composite probe or non-composite probe can be attached to a second plurality of particles.

The first plurality of particles and second plurality of particles are distinguishably encoded for use in certain assay types. The first plurality and second plurality of distinguishably encoded particles are optionally mixed to provide a multiplex assay reagent. Additional particles having additional attached probes can be used in nucleic acid assay described herein in multiplex or separate assay formats.

A method of preparing a substrate-attached composite nucleic acid probe reagent for assay of DNA is provided herein which includes isolating a first nucleic acid sequence which specifically hybridizes to substantially an entire first genomic locus which includes a first terminus of a genomic region of a reference genome. The method includes isolating a second nucleic acid sequence which specifically hybridizes to substantially an entire second genomic locus comprising a second terminus of the genomic region of the reference genome. The first and the second nucleic acid sequences are mixed to produce a composite probe and the composite probe is then bound to a solid substrate, producing a substrate-attached composite nucleic acid probe reagent for assay of nucleic acids, such as genomic DNA.

In a particular option, the first nucleic acid sequence is isolated from a first large-insert vector and the second nucleic acid sequence is isolated from a second large-insert vector. For example, the first nucleic acid sequence is isolated from a first BAC and the second nucleic acid sequence is isolated from a second BAC.

Optionally, the first and the second nucleic acid sequences are amplified prior to or after mixing.

Methods are provided according to particular embodiments which utilize two or more reference samples.

### BRIEF DESCRIPTION OF THE DRAWINGS

FIG. 1 is a flowchart illustrating an embodiment including preparing amplicons from template DNA using two amplification reactions and immobilizing the amplicons as probes onto a set of encoded beads, where the beads in the set all have the same ID code;

FIG. 1A is a flowchart illustrating an embodiment including preparing BAC amplicons from a single BAC clone and immobilizing the amplicons as probes onto a set of encoded beads, generating a bead set where the beads in the set all have the same ID code;

FIG. 2 is a flowchart illustrating an embodiment including mixing m different encoded bead sets, each with its respective immobilized BAC-amplicon probe DNA, together to make a multiplexed encoded bead set;

FIG. 3 is a flowchart illustrating an embodiment including running a multiplexed genomic gain and loss assay on n samples using a multiplexed encoded bead set;

FIG. 3A is a flowchart illustrating an embodiment including running a multiplexed genomic gain and loss assay on n samples using a multiplexed encoded bead set;

FIG. 4 is a schematic diagram of a 96-well SBS-standard microplate, showing example locations of duplicate references and duplicate samples for running an assay on 46 samples in parallel;

FIG. 5 is an example of data generated using a Coriell DNA sample having a trisomy on chromosome 13, sex male;

FIG. 6 is an example of data generated using a Coriell DNA sample having a trisomy on chromosome 18, sex male;

FIG. 7 is an example of data generated using a Coriell DNA sample having a trisomy on chromosome 21, sex female;

FIG. 8 is an example of data generated using a Coriell DNA sample having a 5-copy amplification of the X chromosome;

FIG. 9 is a table displaying the BAC clones used to generate amplicons immobilized onto encoded beads in the example assays, their chromosome and cytoband locations, the sequence of the negative control oligonucleotide, and the bead ID (Luminex bead region) for the bead set to which each amplicon probe is immobilized;

FIG. 10A is a schematic flowchart showing a process for making a composite probe according to an aspect of a process described herein;

FIG. 10B is a schematic flowchart showing a process for making a composite probe according to an aspect of a process described herein;

FIG. 11 is a schematic flowchart showing a process for making a composite probe according to an aspect of a process described herein;

FIG. 12 is a graph of plotted data from a test assay demonstrating the use of a composite probe with a DiGeorge syndrome reference DNA sample;

FIG. 13 is a graph showing ratio data from a Luminex bead array gain-loss assay using two reference genomic DNA samples;

FIG. 14 is a graph showing ratio data from a Luminex bead array gain-loss assay using two reference genomic DNA samples;

FIG. 15 is a flowchart showing a process for making a substrate-attached composite nucleic acid probe according to one aspect described herein;

FIG. 16 is a flowchart showing an embodiment of a genomic gain and loss assay on any number “n” samples using at least one substrate-attached composite nucleic acid probe;

FIG. 17 is a flowchart showing an embodiment of a multiplex genomic DNA gain-loss assay using a multiplex substrate-attached composite nucleic acid probe; and

FIG. 18 is a graph comparing genomic DNA gain-loss assay results using various probe types.

### DETAILED DESCRIPTION OF THE INVENTION

Methods and compositions relating to assays for chromosomal gains and losses are provided herein. Broadly, methods and compositions are described herein which relate to assays of genomic DNA gain and loss using substrate-attached nucleic acid probes.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; and Herdewijn, P. (Ed.), Oligonucleotide Synthesis: Methods and Applications, Methods in Molecular Biology, Humana Press, 2004.

The term “nucleic acid” as used herein refers to RNA or DNA molecules having more than one nucleotide in any form including single-stranded, double-stranded, oligonucleotide or polynucleotide.

Probes

The term “probe” is used herein to refer to a nucleic acid used to identify a target nucleic acid to which the probe specifically binds.

A nucleic acid probe for use in an assay described herein can encompass all or part of a genome of a cell or organism. The nucleic acid probe can encompass DNA representing one or more chromosomes, a portion of a chromosome, a genetic locus, a gene or a portion of a gene. The nucleic acid probe can be in any form, such as an insert in a vector illustratively including a bacterial artificial chromosome, yeast artificial chromosome, human artificial chromosome, cosmid, plasmid, phagemid, phage DNA or fosmid. The nucleic acid probe can be in the form of microdissected chromosomal DNA. Thus, while specific examples described herein refer to BACs as sources of nucleic acid probe DNA, other types of clones such as PACs, YACs, cosmids, fosmids, cDNAs and the like may be used.

In particular applications, nucleic acids are used as template material for amplification and the resulting amplicons, or portions thereof, are used as probes. Nucleic acids for use in generating a nucleic acid probe, sample, or reference are obtained by methods known in the art, for instance, as described in J. Sambrook and D. W. Russell, Molecular Cloning: A Laboratory Manual, Cold Spring Harbor Laboratory Press; 3rd Ed., 2001 or F. M. Ausubel, Ed., Short Protocols in Molecular Biology, Current Protocols; 5th Ed., 2002. Nucleic acids may also be obtained commercially and/or using commercial kits.

Composite Probes

A composite nucleic acid probe is provided according to particular embodiments which includes nucleic acid sequences that specifically hybridize to two or more genomic loci in a genomic region of interest in a reference genome. The genomic region is characterized by a first terminus and a second terminus with an intermediate region of at least 400 kilobases disposed between the termini. The composite nucleic acid probe includes nucleic acid sequences which specifically hybridize to substantially an entire first genomic locus which includes the first terminus of the genomic region and nucleic acid sequences which specifically hybridize to substantially an entire second genomic locus which includes the second terminus. The first genomic locus and second genomic locus each typically include at least about 100 kilobases, and may be larger.

The nucleic acid sequences that specifically hybridize to two or more genomic loci in a genomic region of interest in a reference genome are derived from two or more sources and can be mixed before or after attachment to a solid substrate, producing a “substrate-attached composite” probe.

In some embodiments, nucleic acid sequences that specifically hybridize to two or more genomic loci in a genomic region of a reference genome are mixed, producing a composite nucleic acid probe. The mixed nucleic acid sequences are then attached to a solid substrate, such as particles, producing a “substrate-attached composite” nucleic acid probe.

In further embodiments, nucleic acid sequences that specifically hybridize to two or more genomic loci in a genomic region of a reference genome are not mixed prior to attachment to a solid substrate. Thus, in some embodiments, nucleic acid sequences that specifically hybridize to two or more genomic loci in a genomic region of a reference genome are separately attached to a solid substrate, such as particles, producing a two or more populations of substrate-attached nucleic acid sequences wherein each population of substrate-attached nucleic acid sequences specifically hybridizes to one genomic locus. The two or more populations of substrate-attached nucleic acid sequences are then mixed to produce a substrate-attached composite probe set. In embodiments where the solid substrate is in the form of particles or beads, the composite probe set is referred to as a substrate-attached composite probe set or substrate-attached composite bead set. Each particle or bead in such a set is encoded with the same code such that each particle or bead of the substrate-attached composite probe set is identifiable as a member of that substrate-attached composite probe set and distinguishable from each particle or bead of another substrate-attached probe bead set or particle set.

The nucleic acid sequences which specifically hybridize to two or more genomic loci in a genomic region of a reference genome can be derived from two or more large-insert DNA vectors. For instance, the nucleic acid sequences are derived from two or more large-insert DNA vectors such as bacterial artificial chromosomes, yeast artificial chromosomes, human artificial chromosomes, P1 derived artificial chromosomes (PAC), cosmids, plasmids, phagemids, phage DNA and fosmids. The nucleic acid sequences which specifically hybridize to two or more genomic loci in a genomic region of a reference genome can also be derived from isolated chromosomes and/or isolated chromosome fragments.

In a particular option, the nucleic acid sequences which specifically hybridize to two or more genomic loci in a genomic region of a reference genome are amplicons amplified from templates derived from two or more large-insert DNA vectors, isolated chromosomes, isolated chromosome fragments or a combination of these or other sources of nucleic acids.

In particular embodiments, the nucleic acid sequences which specifically hybridize to two or more genomic loci in a genomic region of a reference genome are provided as oligonucleotides and/or polynucleotides which individually have a length in the range of about 20-250,000 nucleotides, inclusive and which, together, specifically hybridize to substantially an entire first genomic locus which includes the first terminus of the genomic region and to substantially an entire second genomic locus which includes the second terminus of the genomic region.

Thus, a composite probe can include a pool of 2 or more BACs, or the insert DNA derived from 2 or more BACs. Optionally, the pool can include the insert DNA derived from between 4 and about 100 BACs, inclusive. The probe DNA may be extracted from the cultured BACs or in a particular embodiment may be amplicons derived from the BAC DNA, for example by degenerate oligonucleotide primer (DOP) PCR or ligation-mediated PCR. Other large-insert clones, such as PACs, YACs, cosmids, fosmids, etc. can be used instead of BACs. Pools of large numbers of oligonucleotides can also be used.

In a particular aspect, the composite immobilized probes may be DNA derived from sorted chromosomes. Cell-sorting flow cytometers (also known as fluorescence activated cell sorters; FACS) can be used to create pools of sorted chromosomes where the sorting is based on the ratio of signals between stains specific for AT- and CC-rich regions of the genome. DNA extracted from such sorted chromosomes can be amplified by WGA and labeled with a fluorescent dye to be used as chromosome-painting probes in metaphase FISH analysis. Similarly, chromosome arm painting probes can be prepared by amplifying the DNA from sorted chromosome arms separated from metaphase spreads by laser-capture microdissection. Immobilized whole-chromosome or chromosome-arm probes immobilized as microarray spots or onto encoded particles produce hybridization ratio signals representing the average gain or loss across the whole chromosome or arm, respectively, in a manner similar to a pool of a very large number of BACs that span an entire chromosome or arm.

Composite probes have utility in a variety of roles in a multiplex genomic gain-loss assay, as exemplified in a multiplex genomic gain-loss assay described herein. These include chromosome enumeration (detection of aneuploidy), detection of microdeletion or other syndromes, or as controls. Using composite probes as controls to determine the normal autosomal response (the nominal sample/reference ratio=1.0 level) allows averaging of response over a larger span of the genome to reduce ratio noise caused by normal copy number variations between individuals.

Processes for Making Composite Probes

A method of preparing a substrate-attached composite nucleic acid probe reagent for assay of DNA is provided herein. A first nucleic acid sequence is isolated which specifically hybridizes to substantially an entire first genomic locus which includes a first terminus of a genomic region of a reference genome. Further, at least a second nucleic acid sequence is isolated which specifically hybridizes to substantially an entire second genomic locus comprising a second terminus of the genomic region of the reference genome. The term “isolated” when used in reference to nucleic acids refers to nucleic acids substantially separated from other substances with which they are naturally found, such as cells, proteins and other nucleic acids.

In some embodiments, the first and the second nucleic acid sequences are mixed to produce a composite probe and the composite probe is then bound to a solid substrate, producing a substrate-attached composite nucleic acid probe reagent for assay of nucleic acids, such as genomic DNA.

In further embodiments, the first and the second nucleic acid sequences are kept separate and bound to solid substrates. The resulting substrate-attached probes are then mixed, producing a reagent for assay of nucleic acids, such as genomic DNA.

In a particular option, the first nucleic acid sequence is isolated from a first large-insert vector and the second nucleic acid sequence is isolated from a second large-insert vector. For example, the first nucleic acid sequence is a DNA insert isolated from a first BAC and the second nucleic acid sequence is a DNA insert isolated from a second BAC.

In a further option, the first nucleic acid sequence is a human genomic DNA insert isolated from a first BAC and the second nucleic acid sequence is a human genomic DNA insert isolated from a second BAC.

Optionally, the nucleic acid sequences are amplified. In the case of composite substrate attached probes, the nucleic acid sequences can be amplified prior to mixing, or after mixing. For example, the first and the second, or more, nucleic acid sequences are amplified prior to or after mixing.

The number of genomic loci targeted by nucleic acid sequences in a composite probe is not limited to two and may be three, four or more, such as about 4-100, or more. Thus, the number of particular nucleic acid sequences which specifically hybridize to genomic loci is likewise not limited to two and may be three, four or more, such as about 4-100, or more.

An example of a composite probe is a composite probe made using insert DNA from five BACs mapping to cytoband 22p11.2 corresponding to the DiGeorge microdeletion syndrome. The center loci of the five BACs used span about 0.45 megabases (445 kilobases), the total span accounting for the 175 kb typical length of the BACs is a little over 600 kilobases. The isolated insert DNA from each of the five BACs is amplified and the resulting amplicons are pooled to produce a composite probe. The composite probe is then bound to a set of Luminex encoded multiplex microspheres, all having the same bead encoding identification to produce a substrate-attached composite probe set, for use as a 22p11.2 cytoband probe in a multiplex genomic gain-loss assay.

A further example of a composite probe is a composite probe made using insert DNA from five BACs mapping to cytoband 22p11.2 corresponding to the DiGeorge microdeletion syndrome such as described above. The isolated insert DNA from each of the five BACs is amplified and the resulting five sets of amplicons corresponding to the five BACs are bound to five sets of Luminex encoded multiplex microspheres, all having the same bead encoding identification. The resulting five populations of substrate-attached amplicons are then mixed to produce a substrate-attached composite probe set for use as a 22p11.2 cytoband probe in a multiplex genomic gain-loss assay.

Thus, a particular example, 2, 3, 4, 5 or more, such as 6-100, or more, inserts from BAC, or other large insert vectors, can be used as nucleic acid sequences which specifically hybridize to particular genomic loci in a defined genomic region.

Substrates

A solid substrate, which includes semi-solid substrate, for attachment of a probe, including a composite probe, can be any of various materials such as glass; plastic, such as polypropylene, polystyrene, nylon; paper; silicon; nitrocellulose; or any other material to which a nucleic acid can be attached for use in an assay. The substrate can be in any of various forms or shapes, including planar, such as silicon chips and glass plates; and three-dimensional, such as particles, microtiter plates, microtiter wells, pins, fibers and the like.

In particular aspects, a solid substrate to which a probe is attached is a particle.

Particles to which a probe is bound can be any solid or semi-solid particles to which a probe can be attached, which are suitable for a nucleic acid hybridization assay and which are stable and insoluble under hybridization and detection conditions. The particles can be of any shape, such as cylindrical, spherical, and so forth, size, composition, or physiochemical characteristics. The particle size or composition can be chosen so that the particle can be separated from fluid, e.g., on a filter with a particular pore size or by some other physical property, e.g., a magnetic property.

Microparticles, such as microbeads, used can have a diameter of less than one millimeter, for example, a size ranging from about 0.1 to about 1,000 micrometers in diameter, inclusive, such as about 3-25 microns in diameter, inclusive, or about 5-10 microns in diameter, inclusive. Nanoparticles, such as nanobeads used can have a diameter from about 1 nanometer (nm) to about 100,000 nm in diameter, inclusive, for example, a size ranging from about 10-1,000 nm, inclusive, or for example, a size ranging from 200-500 nm, inclusive. In certain embodiments, particles used are beads, particularly microbeads and nanobeads.

Particles are illustratively organic or inorganic particles, such as glass or metal and can be particles of a synthetic or naturally occurring polymer, such as polystyrene, polycarbonate, silicon, nylon, cellulose, agarose, dextran, and polyacrylamide. Particles are latex beads in particular embodiments.

Particles used include functional groups for binding to nucleic acids in particular embodiments. For example, particles can include carboxyl, amine, amino, carboxylate, halide, ester, alcohol, carbamide, aldehyde, chloromethyl, sulfur oxide, nitrogen oxide, epoxy and/or tosyl functional groups. Functional groups of particles, modification thereof and binding of a chemical moiety, such as a nucleic acid, thereto are known in the art, for example as described in Fitch, R. M., Polymer Colloids: A Comprehensive Introduction, Academic Press, 1997. U.S. Pat. No. 6,048,695 describes an exemplary method for attaching nucleic acid probes, such as amplicons, to a substrate, such as particles. In a further particular example, 1-Ethyl-3-[3-dimethylaminopropyl]carbodiimide hydrochloride, EDC or EDAC chemistry, can be used to attach nucleic acids to encoded particles.

Encoded particles are particles which are distinguishable from other particles based on a characteristic illustratively including an optical property such as color, reflective index and/or an imprinted or otherwise optically detectable pattern. For example, the particles may be encoded using optical, chemical, physical, or electronic tags. Encoded particles can contain or be attached to, one or more fluorophores which are distinguishable, for instance, by excitation and/or emission wavelength, emission intensity, excited state lifetime or a combination of these or other optical characteristics. Optical bar codes can be used to encode particles.

In particular embodiments, each particle of a particle set is encoded with the same code such that each particle of a particle set is distinguishable from each particle of another particle set. In further embodiments, two or more codes can be used for a single particle set. Each particle can include a unique code, for example. In certain embodiments, particle encoding includes a code other than or in addition to, association of a particle and a nucleic acid probe specific for genomic DNA.

In particular embodiments, the code is embedded, for example, within the interior of the particle, or otherwise attached to the particle in a manner that is stable through hybridization and analysis. The code can be provided by any detectable means, such as by holographic encoding, by a fluorescence property, color, shape, size, light emission, quantum dot emission and the like to identify particle and thus the capture probes immobilized thereto. In some embodiments, the code is other than one provided by a nucleic acid.

One exemplary platform utilizes mixtures of fluorescent dyes impregnated into polymer particles as the means to identify each member of a particle set to which a specific capture probe has been immobilized. Another exemplary platform uses holographic barcodes to identify cylindrical glass particles. For example, Chandler et al. (U.S. Pat. No. 5,981,180) describes a particle-based system in which different particle types are encoded by mixtures of various proportions of two or more fluorescent dyes impregnated into polymer particles. Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. U.S. Patent Application Publications 20040179267, 20040132205, 20040130786, 20040130761, 20040126875, 20040125424, and 20040075907 describe exemplary particles encoded by holographic barcodes. U.S. Pat. No. 6,916,661 describes polymeric microparticles that are associated with nanoparticles that have dyes that provide a code for the particles

While an embodiment described in detail herein utilizes the Luminex encoded bead platform, other types of encoded particle assay platforms may be used, such as the VeraCode beads and BeadXpress system (Illumina Inc., San Diego Calif.), xMAP 3D (Luminex) and the like. Magnetic Luminex beads can be used which allow wash steps to be performed with plate magnets and pipetting rather than with filter plates and a vacuum manifold. Each of these platforms are typically provided as carboxyl beads but may also be configured to include a different coupling chemistry, such as amino-silane.

Binding to Substrate

Binding of the nucleic acid probes to a substrate is achieved by any of various methods effective to bond a nucleic acid to a solid or semi-solid substrate, illustratively including adsorption and chemical bonding. The nucleic acids can be bonded directly to the material of the encoded particles or indirectly bonded to the encoded particles, for example, via bonding to a coating or linker disposed on the particles. Nucleic acids can be synthesized, and/or modified once synthesized, to include a functional group for use in bonding the nucleic acids to particles. For example, the nucleic acids sequences used as probes can include carboxyl, amine, amino, carboxylate, halide, ester, alcohol, carbamide, aldehyde, chlorom ethyl, sulfur oxide, nitrogen oxide, epoxy and/or tosyl functional groups.

Probes, including composite probes, attached to a substrate can be single-stranded and/or double-stranded nucleic acids. In particular embodiments, where double-stranded nucleic acids are bound, they are denatured and rendered single stranded after immobilization to the substrate for preparation for use in certain embodiments of assay methods. Optionally, double stranded nucleic acid probes are denatured prior to immobilization and the single stranded nucleic acids are then bound to the substrate.

Amplicon Reagent Compositions

In particular embodiments, a reagent for assaying nucleic acids is provided which includes a plurality of encoded particles having attached amplicons as probes.

In further particular embodiments, the attached amplicons are amplified from more than one template, making up a composite probe.

In certain embodiments, the amplicons attached to the plurality of encoded particles each include a nucleic acid sequence identical or completely complementary to a portion of a template genomic nucleic acid and together the amplicons represent substantially the entire template genomic nucleic acid.

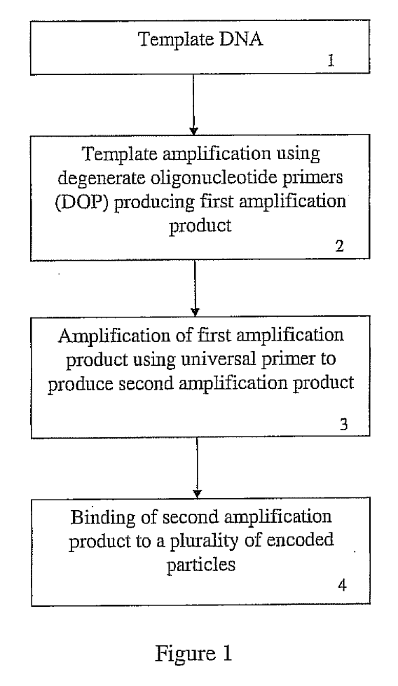

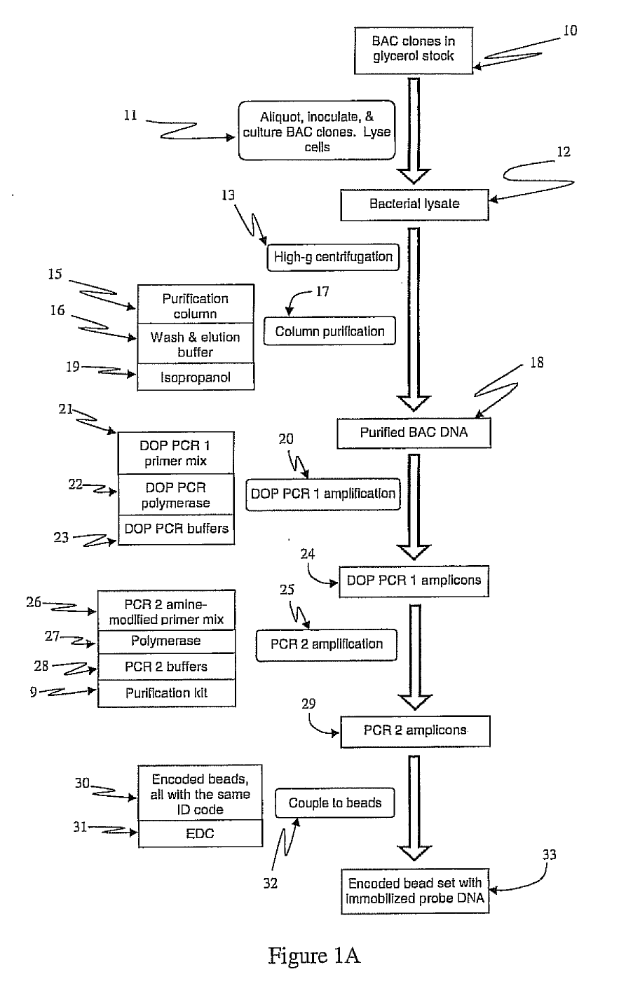

FIG. 1 illustrates an embodiment of a process for making a reagent for assaying genomic DNA. As indicated in FIG. 1, a template nucleic acid is provided, 1. The template is amplified, 2, in a first amplification reaction using degenerate oligonucleotide primers (DOP) to produce a first amplification product.

The template nucleic acid can be any nucleic acid capable of being copied using a nucleic acid amplification method.

The template DNA for this first amplification reaction is optionally genomic DNA, typically having a size in the range of about 20-300 kb, although the template can be smaller or larger. The term “genomic” refers to DNA of the genome of a cell or organism and includes DNA isolated directly from a cell or organism, such as microdissected chromosomal DNA, as well as DNA copied from DNA of the genome of a cell or organism, such as cloned DNA. The template DNA can encompass all or part of a genome of a cell or organism. The template DNA can encompass DNA representing one or more chromosomes, a portion of a chromosome, a genetic locus, a gene or a portion of a gene. The template DNA can be in any form, such as an insert in a vector illustratively including a bacterial artificial chromosome, yeast artificial chromosome, human artificial chromosome, cosmid, plasmid, phagemid, phage DNA or fosmid. Template DNA can be in the form of microdissected chromosomal DNA. Thus, while specific examples described herein refer to BACs as sources of template DNA, other types of clones such as PACs, YACs, cosmids, fosmids, cDNAs and the like may be used.

Multiple templates from any of these or other sources can be amplified together or separately and the combined amplicons from the multiple templates are a composite probe according to particular embodiments.

Template genomic DNA is obtained by methods known in the art, for instance, as described in J. Sambrook and D. W. Russell, Molecular Cloning: A Laboratory Manual, Cold Spring Harbor Laboratory Press; 3rd Ed., 2001 or F. M. Ausubel, Ed., Short Protocols in Molecular Biology, Current Protocols; 5th Ed., 2002. Template DNA may also be obtained commercially and/or using commercial kits for isolation of genomic DNA.

Amplification of template DNA is achieved using an in vitro amplification method. The term “amplification method” refers to a method or technique for copying a template nucleic acid, thereby producing nucleic acids including copies of all or a portion of the template nucleic acid, the produced nucleic acids also termed amplicons.

Amplicons optionally contain nucleic acid sequences present in the primers and not present in the original DNA template. Such primer-derived nucleic acids add functionality such as primer binding sites for additional amplification reactions and/or a functional group for chemical bonding to a substrate.

Amplification methods illustratively including PCR, ligation-mediated PCR (LM-PCR), phi-29 PCR, and other nucleic acid amplification methods, for instance, as described in C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

Many combinations of particular DNA template sources and nucleic acid amplification methods may be used.

The term “oligonucleotide primer” refers to a nucleic acid that is capable of acting as a site of initiation of synthesis of a primer extension product under appropriate reaction conditions. An oligonucleotide primer is typically about 10-30 contiguous nucleotides in length. An oligonucleotide primer is completely or substantially complementary to a region of a template nucleic acid such that, under hybridization conditions, the oligonucleotide primer anneals to the complementary region of the template nucleic acid. Appropriate reactions conditions for synthesis of a primer extension product include presence of suitable reaction components including, but not limited to, a polymerase and nucleotide triphosphates. Design of oligonucleotide primers suitable for use in amplification reactions is well known in the art, for instance as described in A. Yuryev et al., PCR Primer Design, Humana Press, 2007.

The term “degenerate oligonucleotide primer” refers to a primer which includes a nucleic acid having a random or semi-random nucleotide sequence. Design of degenerate oligonucleotide primers suitable for particular nucleic acid amplification reactions is well known in the art for instance as described in A. Yuryev et al., PCR Primer Design, Humana Press, 2007. Random or semi-random nucleotide sequences having about 5-8 nucleotides can be used. In further embodiments, random or semi-random nucleotide hexamers are included in degenerate oligonucleotide primers used in the first amplification.

The degenerate oligonucleotide primers used in particular embodiments each include a 5′ constant DNA segment, an intermediate random DNA segment and a 3′ anchor segment, for example as described in Fiegler et al., Genes Chromosomes Cancer, 36(4):361-74, 2003; and Telenius, et al., Genomics 13:718-25, 1992. The 5′ constant DNA segment optionally has the same nucleotide sequence in all of the DOPs. The 3′ anchor segment optionally has a nucleotide sequence determined to have a desired frequency of occurrence in the template nucleic acid. Analysis of frequency of occurrence of a particular nucleic acid sequence is well known in the art, for example, as described in Milosavljevic, A. and Jurka, J., 1993, Comput. Applic. Biosci., 9:407-411; Pesole, G. et al., 1992, Nucleic Acids, Res., 20:2871-2875; and Hutchinson, G. B., 1996, Comput. Appl. Biosci., 12:391-398.

In particular embodiments the DOPs include about 17-25 contiguous nucleotides, of which about 7-12 contiguous nucleotides are included in the 5′ constant DNA segment, about 5-8 contiguous nucleotides are included in the random DNA segment and about 5-8 contiguous nucleotides are included in the 3′ anchor segment.

The first amplification reaction yields a first reaction product containing a plurality of amplicons. Each individual amplicon in the first reaction product includes a DNA sequence identical or completely complementary to a random portion of the DNA template and a DNA sequence identical to the 5′ constant DNA sequence of the first reaction primers.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100%, or completely, complementary to a region of the nucleotide sequence 5′-TTAGCTGG-3′.

Referring to FIG. 1, a second amplification reaction, 3, is performed using the first reaction product amplicons as template DNA. The second amplification reaction, 3, includes a “universal” oligonucleotide primer, so-called since the universal primer is identical or completely complementary to the 5′ constant DNA segment of the DOP used in the first amplification reaction. A universal oligonucleotide primer includes the 5′ constant DNA segment of the DOP used in the first amplification reaction positioned at the 3′ end of the universal primer. A universal oligonucleotide primer optionally includes additional contiguous nucleotides at the 5′ end of the primer.

In a particular option, a universal oligonucleotide primer includes a functional group at the 5′ terminus of the primer for attachment of the amplicons resulting from the second amplification reaction to an encoded solid or semi-solid substrate such as encoded particles. For example, the universal oligonucleotide primers include an amine group at the 5′ terminus of the primer. In a further option, amplicons resulting from the second amplification reaction can be modified to include a functional group for bonding to a solid or semi-solid substrate Modification of a nucleic acid to include a functional group capable of bonding to a solid or semi-solid substrate is well known in the art.

In particular embodiments, each individual amplicon attached to a particle includes a DNA segment identical to a random portion of the template DNA sequence. Each individual amplicon also contains a constant DNA segment contiguous with the DNA segment identical to a random portion of the template DNA sequence. The constant DNA segment of the amplicon optionally includes a terminal functional group for attachment of the amplicon to an encoded particle. In a particular embodiment, the constant DNA segment of the amplicon includes a 5′ terminal amine group for attachment of the amplicon to an encoded particle.

As shown in FIG. 1, the amplicons of the second reaction product are immobilized, 4, on a first plurality of encoded particles. Binding of the amplicons of the second amplification reaction to the encoded particles is achieved by any of various methods effective to bond a nucleic acid to a solid or semi-solid substrate, illustratively including adsorption and chemical bonding. The amplicons can be bonded directly to the material of the encoded particles or indirectly bonded to the encoded particles, for example, via bonding to a coating or linker disposed on the particles. Amplicons can be synthesized, and/or modified once synthesized, to include a functional group for use in bonding the amplicons to particles. For example, amplicons can include carboxyl, amine, amino, carboxylate, halide, ester, alcohol, carbamide, aldehyde, chloromethyl, sulfur oxide, nitrogen oxide, epoxy and/or tosyl functional groups.

In general, the amplicons which are the product of the second amplification reaction are double stranded and the double stranded amplicons are attached to the particles. Thus, both strands of the double stranded amplicons are represented on each particle. The amplicons are denatured and rendered single stranded after immobilization to the particles for preparation for use in particular embodiments of assay methods. Optionally, double stranded amplicons are denatured prior to immobilization and the single stranded amplicons are then bound to particles.

As described, each individual amplicon of both the first and second amplification reactions contains a nucleic acid sequence identical to a random portion of the template DNA sequence such that the amplicons produced by the first amplification reaction together represent substantially the entire template DNA sequence and the amplicons produced by the second amplification reaction together represent substantially the entire template DNA sequence.

Encoded particles having bound amplicons which are the product of a second amplification reaction and which together represent substantially the entire genomic DNA sequence used as a template in the first amplification reaction are a first particle set and a first reagent for assaying genomic DNA.

In particular embodiments, each individual amplicon attached to a particle has a length in the range of about 500-1200 nucleotides, inclusive. Thus, a relatively large template nucleic acid is represented substantially entirely on a set of encoded particles by the attached relatively smaller amplicons amplified from the template.

As noted above, each particle set includes encoded particles having bound amplicons which are the product of a second amplification reaction and which together represent substantially the entire genomic DNA sequence used as a template in a first amplification reaction. The number of particles including amplicons which is sufficient to together represent substantially the entire genomic DNA sequence used as a template in the first amplification reaction depends on a number of factors such as the size of the template, the size of the amplicons and the number of binding sites available for binding an amplicon on a particle. In general, the number of particles sufficient to together represent substantially the entire genomic DNA sequence used as a template in the first amplification reaction is in the range of about 1-10,000, inclusive.

Additional particle sets are generated by amplification using a second genomic DNA template and binding the amplicons which are the reaction product of a second amplification reaction as described above to a second plurality of encoded particles. The second plurality of encoded particles is delectably different than the first plurality of encoded particles, thereby generating a second encoded particle set and a second reagent for assaying genomic DNA.

Similarly, a third or subsequent genomic DNA template is used to generate the reaction product of an amplification reaction and the reaction product is bound to a third or subsequent plurality of encoded particles. Each of the third or subsequent plurality of encoded particles is detectably different than each other plurality of encoded particles, yielding a third or subsequent encoded particle set and a third or subsequent reagent for assaying genomic DNA.

Multiplex Reagent

A multiplex reagent for assaying genomic DNA is provided according to certain embodiments which includes a mixture of two or more particle sets. The individual encoded particles of each encoded particle set are detectably distinguishable from individual encoded particles of each other encoded particle set in particular embodiments.

In particular embodiments, at least one particle set includes a composite probe. Optionally, more than one particle set include a composite probe.

In particular embodiments, each encoded particle set has attached amplicons which are the product of a second amplification reaction as described herein and which together represent substantially the entire genomic DNA sequence used as a template in a first amplification reaction, wherein a different genomic template is represented by amplicons attached to each other encoded particle set.

A multiplex reagent according to a specific embodiment includes a first encoded particle set having attached amplicons which together represent substantially an entire template DNA sequence inserted in the first bacterial artificial chromosome and a second encoded particle set having attached amplicons which together represent substantially an entire template DNA sequence inserted in the second bacterial artificial chromosome.

For example, a first encoded particle set has attached amplicons including nucleic acid sequences identical to a portion of human chromosome 13 DNA and a second encoded particle set has attached amplicons including nucleic acid sequences identical to a portion of chromosome 18 human DNA. Third or subsequent encoded particle set have attached amplicons including nucleic acid sequences identical to human DNA from another chromosome or another non-overlapping region of a chromosome.

A multiplex reagent described herein allows for simultaneous assay of multiple targets, such as multiple genomic loci, in a single assay.

A multiplex reagent for assaying genomic DNA is generated by mixing at least a first encoded particle set and a second encoded particle set.

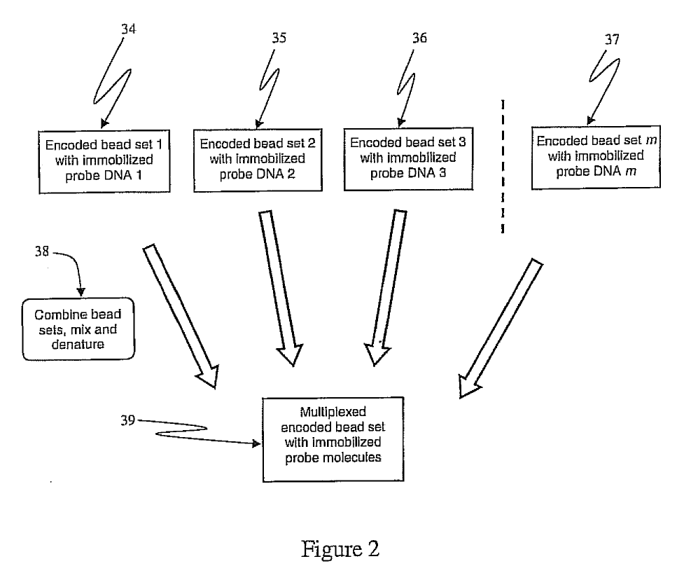

FIG. 2 illustrates an embodiment of a method of generating a multiplex reagent. As indicated in the figure, any number, “in” of encoded particle sets can be included in the multiplex reagent. Thus, for example, “m” can be at least 2, 3, 4, 5, 10, 15, 20, 25, 30, 35, 40, 45, 50, 55, 60, 65, 70, 75, 80, 85, 90, 95, 100 or 200 different encoded particle sets. A set of encoded particles having bound amplicons is combined with one or more additional sets of encoded particles having bound amplicons to generate a multiplex reagent for assay of genomic gain and loss in a sample.

In a particular embodiment, a set of encoded particles including a bound composite probe is combined with one or more additional sets of encoded particles having bound composite or non-composite probes to generate a multiplex reagent for assay of genomic gain and loss in a sample.

In some embodiments, a substrate-attached composite probe set is combined with one or more other substrate-attached composite probe sets to generate a multiplex reagent for assay of genomic gain and loss in a sample.

Assay Methods

A method of assaying a DNA sample is provided according to particular embodiments which includes providing a substrate-attached composite nucleic acid probe. The substrate-attached composite nucleic acid probe includes nucleic acid sequences that specifically hybridize to two or more genomic loci in a genomic region of a reference genome. The genomic region is characterized by a first terminus and a second terminus with an intermediate region of at least 400 kilobases disposed between the termini. The substrate-attached composite nucleic acid probe includes nucleic acid sequences which specifically hybridize to substantially an entire first genomic locus which includes the first terminus of the genomic region and nucleic acid sequences which specifically hybridize to substantially an entire second genomic locus which includes the second terminus. The first genomic locus and second genomic locus each typically include at least about 100 kilobases. Optionally, nucleic acid sequences of the substrate-attached composite probe specifically hybridize to additional loci within the genomic region. In a further option, the reference genome is one or more human genomes.

The substrate-attached composite nucleic acid probe is hybridized with sample genomic DNA at a stringency sufficient to achieve specific hybridization. The substrate-attached composite nucleic acid probe is also hybridized with reference genomic DNA under the same or similar conditions to allow for comparison.

A first signal indicating specific hybridization of the substrate-attached composite nucleic acid probe with the sample genomic DNA is detected along with a second signal indicating specific hybridization of the substrate-attached composite nucleic acid probe with the reference genomic DNA. The first signal and the second signal are compared to detect differences between the first and second signals, indicative of differences between the sample DNA and the reference DNA.

Particular embodiments of methods of assaying sample nucleic acid include providing a multiplex reagent including a mixture of two or more encoded particle sets encoded such that each particle of each encoded particle set is detectably distinguishable from each particle of each other encoded particle set. The encoded particles include attached nucleic acid sequences which specifically hybridize to at least one genomic locus of a reference genome, and at least one encoded particle set includes an attached composite nucleic acid probe.

The multiplex reagent is hybridized with sample genomic nucleic acid and with reference nucleic acid, together or in parallel.

A first signal is detected which indicates specific hybridization of the attached nucleic acid sequences with detectably labeled sample nucleic acid and a second signal is detected indicating specific hybridization of the attached nucleic acid sequences with detectably labeled reference nucleic acid. The encoded particles are then identified so as to associate particle encoding with the first signal or with the second signal. The first signal and the second signal for each encoded particle set are then compared and differences in the first and second signals are indicative of differences between the sample and reference nucleic acids.

In particular embodiments, methods of assaying genomic DNA include providing encoded particles having attached amplicons which together represent substantially an entire template genomic nucleic acid. In particular embodiments, encoded particles having attached amplicons are provided which together represent more than one copy of substantially an entire template genomic nucleic acid.

In particular embodiments, a sample of genomic DNA to be assayed for genomic gain and/or loss is labeled with a detectable label. Reference DNA is also labeled with a detectable label for comparison to the sample DNA. The sample and reference DNA can be labeled with the same or different detectable labels depending on the assay configuration used. For example, sample and reference DNA labeled with different detectable labels can be used together in the same container for hybridization with amplicons attached to encoded particles in particular embodiments. In further embodiments, sample and reference DNA labeled with the same detectable labels can be used in separate containers for hybridization with amplicons attached to particles.

The term “detectable label” refers to any atom or moiety that can provide a detectable signal and which can be attached to a nucleic acid. Examples of such detectable labels include fluorescent moieties, chemiluminescent moieties, bioluminescent moieties, ligands, magnetic particles, enzymes, enzyme substrates, radioisotopes and chromophores.

Any of various methods of labeling sample and reference nucleic acids, such as DNA, may be used in the assay, such as nick translation or chemical labeling of the nucleic acids. For example, a detectable label can be introduced by polymerization using nucleotides that include at least some modified nucleotides, such as nucleotides modified to include biotin, digoxygenin, fluorescein, or cyanine. In some embodiments, the detectable label is introduced by random-priming and polymerization. Other examples include nick translation (Roche Applied Science, Indianapolis Ind.; Invitrogen, Carlsbad Calif.) and chemical labeling (Kreatech ULS, Amsterdam NL). Detectable labeling of nucleic acids is well known in the art and any labeling method appropriate for labeling nucleic acids, such as genomic DNA, can be used.

In yet another embodiment, covalent labeling of sample and reference nucleic acids, such as DNA, individually with a detectable label is avoided. For example, unlabeled genomic DNA samples are hybridized to the amplicons immobilized to the encoded particles. Pre-labeled reporter sequences are also hybridized to the amplicon-sample DNA complexes and amplicon-reference DNA complexes at sequences adjacent to but not overlapping the sequences of the capture probes of the amplicons. These labeled reporter sequences can be hybridized in the same or in a different hybridization reaction. In this manner the labeled reporter sequences can be manufactured in bulk in a larger-scale environment, lowering the cost per assay compared to individually labeling each sample at the time of the assay.

The “sample” and “reference” nucleic acids, such as genomic DNA, can be obtained from any suitable source. Particular methods described herein involve using sample genomic DNA from an individual subject. Genomic sample and/or reference DNA can be extracted from almost any tissue including, but not limited to, blood, amniotic fluid, solid tumors, organ biopsies, cheek swabs, chorionic villae, blastocysts and blastomeres, products of conception, saliva, urine and the like. Archived samples extracted from formalin-fixed, paraffin-embedded (FFPE) pathology samples are also sources of sample genomic DNA assayed by this method. Sample and/or reference genomic DNA can also be obtained from in vitro sources such as cell lines. Methods of obtaining genomic DNA from these or other sources are well known in the art.

In particular embodiments, reference DNA is characterized with respect to a particular characteristic of the sample DNA to be assayed. For example, where sample DNA is to be assayed to detect duplication of a particular gene or chromosomal locus, the reference DNA is characterized so that it is known how many copies of the gene or locus are contained in the reference DNA. In general, sample and reference DNA from the same species are used.

Reference DNA can be a pooled mixture of genomic DNA derived from a plurality of normal subjects, particularly human subjects, of the same gender. DNA pooled from a plurality of normal subjects can be obtained commercially.

In some embodiments, more than one reference DNA is used and additional information is obtained in a process using additional reference DNAs.

Thus, for example, in a particular embodiment, two reference genomic DNA samples are compared to a test sample of genomic DNA. A first reference genomic DNA obtained from a male subject and a second reference genomic DNA obtained from a female subject are compared to a test sample of genomic DNA obtained from a subject whose gender is to be determined, such as a prenatal fetus.

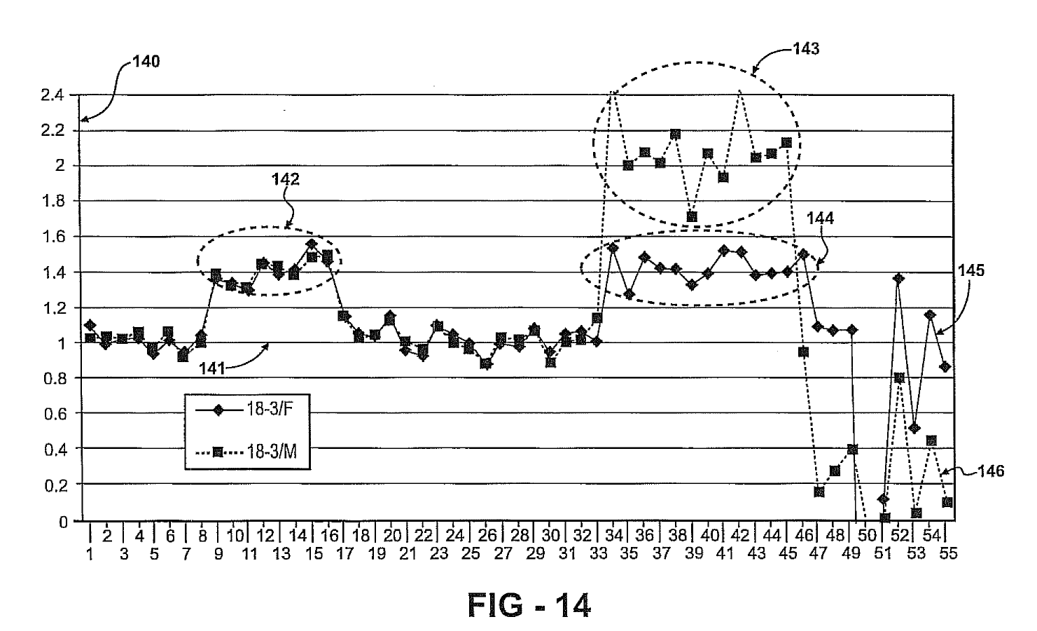

A particular characteristic of an assay of the present invention which includes comparison of at least two reference genomic DNA samples to a test sample of genomic DNA is decreased ambiguity and increased confidence in the assay results. For example, as shown in FIG. 14, an ambiguous result which would have been achieved with only a male-specific reference is clarified when the assay is performed using both a male-specific reference and a female-specific reference.

An assay described herein can be used to detect or characterize disorders associated with chromosomal gains or losses. Constitutional, or inborn, disorders include trisomies of entire chromosomes, amplifications or deletions of smaller genomic loci (approximately 200 kilobases to 20 megabases), and amplifications or deletions in the sub-telomeric or centromeric regions. Various cancers are also characterized by chromosomal gains and losses that may correlate with type, stage, drug resistance, or therapy response. Laboratory cell lines, including stem-cell lines, may be characterized for chromosomal stability using the present method.

Thus, two or more reference genomic DNA samples can be used which represent different stages of a progressive disease, condition or disorder which affects genomic DNA and the two or more references are compared with a test sample of genomic DNA of an individual subject whose condition relative to the references is to be determined. For example, various cancers, other disorders and/or age are associated with progressive deletion or genomic DNA, such as mitochondrial DNA deletions and telomere shortening.

While methods and compositions are described herein primarily with reference to nucleic acids derived from humans, it is appreciated that methods and compositions described herein may be used to assay sample genomic DNA from any of various organisms including, but not limited to, non-human primates, rodents, rabbits, dogs, cats, horses, cattle, pigs, goats and sheep. Non-mammalian sources of sample DNA can also be assayed, illustratively including fish and other aquatic organisms, birds, poultry, bacteria, viruses, plants, insects, reptiles, amphibians, fingi and mycobacteria. Similarly, reference DNA can be human DNA or DNA from any of various organisms including, but not limited to, non-human primates, rodents, rabbits, dogs, cats, horses, cattle, pigs, goats, sheep and non-mammalian sources illustratively including fish and other aquatic organisms, birds, poultry, bacteria, viruses, plants, insects, reptiles, amphibians, fungi and mycobacteria.

The substrate-attached nucleic acid probes are hybridized with detectably labeled sample genomic DNA of an individual subject so as to achieve specific hybridization of the substrate-attached nucleic acid probes and the sample and/or reference nucleic acids.

In particular embodiments of assays described herein, amplicons attached to the encoded particles are hybridized with detectably labeled sample genomic DNA of an individual subject so as to achieve specific hybridization of the amplicon DNA and the detectably labeled sample genomic DNA. In addition, DNA sequences attached to the encoded particles are hybridized with detectably labeled reference genomic DNA so as to achieve specific hybridization of the amplicon DNA and the detectably labeled reference genomic DNA.

The terms “hybridization” and “hybridized” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize. The terms “specific hybridization” and “specifically hybridizes” refer to hybridization of a particular nucleic acid to a target nucleic acid without substantial hybridization to nucleic acids other than the target nucleic acid in a sample.

Assays described can be performed in any suitable container. In particular embodiments, for example, where multiple samples are to be assayed, a multi-chamber container can be used. Multi-chamber containers illustratively include multi-depression substrates such as slides, silicon chips or trays. In some embodiments, each sample is disposed in a different well of a multi-well plate. For example, a multi-well plate can be a 96-well, 384-well, 1024-well or 536-well assay plate.

Further included is detection of a first signal indicating specific hybridization of the attached DNA sequences with detectably labeled genomic DNA of an individual subject and detection of a second signal indicating specific hybridization of the attached DNA sequences with detectably labeled reference genomic DNA.

Any appropriate method, illustratively including spectroscopic, optical, photochemical, biochemical, enzymatic, electrical and/or immunochemical is used to detect a signal in an assay described herein.

Signals that are indicative of the extent of hybridization can be detected, for each particle, by evaluating signal from one or more detectable labels. Particles are typically evaluated individually. For example, the particles can be passed through a flow cytometer. Exemplary flow cytometers include the Coulter Elite-ESP flow cytometer, or FACScan™. flow cytometer available from Beckkman Coulter, Inc. (Fullerton Calif.) and the MOFLO™. flow cytometer available from Cytomation, Inc., Fort Collins, Colo. In addition to flow cytometry, a centrifuge may be used as the instrument to separate and classify the particles. A suitable system is that described in U.S. Pat. No. 5,926,387. In addition to flow cytometry and centrifugation, a free-flow electrophoresis apparatus may be used as the instrument to separate and classify the particles. A suitable system is that described in U.S. Pat. No. 4,310,408. The particles may also be placed on a surface and scanned or imaged.

In certain embodiments, a first signal is detected indicating specific hybridization of the substrate-attached nucleic acid sequences with detectably labeled sample nucleic acids, such as genomic DNA of an individual subject. A second signal is also detected indicating specific hybridization of the substrate-attached nucleic acid sequences with detectably labeled reference genomic DNA.

In further embodiments, a first signal is detected indicating specific hybridization of the encoded particle attached DNA sequences with detectably labeled genomic DNA of an individual subject. A second signal is also detected indicating specific hybridization of the encoded particle attached DNA sequences with detectably labeled reference genomic DNA.

The first signal and the second signal are compared, yielding information about the sample and reference nucleic acids. In certain embodiments, first signal and the second signal are compared, yielding information about the genomic DNA of the individual subject compared to the reference genomic DNA.

In particular embodiments, a ratio of the signals from the detectable labels of the reference DNA and the sample DNA hybridized to the amplicons of one or more particle sets is used to evaluate differences between the sample and reference DNA, indicative, for instance, of genomic gain and/or loss.

In certain embodiments, the reference DNA and the sample DNA are hybridized to the probes, such as amplicons, of one or more particle sets in the same container, such as a well of a multi-well plate. After hybridization, the two labels are analysed together, i.e. both detectable labels are detected in the hybridized material or the hybridized material is divided into two (or more) portions and each portion is evaluated separately to detect the detectable labels. Results from the evaluation can be used to provide the ratio of signals from the two detectable labels. This approach allows use of competitive hybridization to normalize any variation between assays: both of the reference and experimental samples are assayed simultaneously in the same vessel mixed with the same particles.

Optionally, the detectably labeled reference DNA and the detectably labeled sample DNA are hybridized to one or more particle sets in the different containers, such as different wells of a multi-well plate. In a further example, the detectably labeled reference DNA and the detectably labeled sample DNA are hybridized to one or more probes attached at different locations on a planar array. A ratio of signals from the two detectable labels can be obtained to evaluate differences between the sample DNA and reference DNA. When this approach is utilized, a single reference sample can be shared between several or many experimental samples. For experiments involving multiple samples per day there can be a savings on reagent cost and labor by avoiding the labeling of multiple duplicate normal samples. Also it is unnecessary to manipulate the sample to obtain different portions for separate analysis. Each sample can be evaluated only once.

Encoded particles are identified by their encoded information so as to associate particle encoding with the first signal and with the second signal in particular embodiments. Thus, for example, first and second signals are associated with encoded particles of a first encoded particle set containing human DNA from chromosome 13. The first signal and the second signal associated with the first encoded particle set are compared, yielding information about the chromosome 13 DNA of the individual subject compared to the chromosome 13 reference DNA. Similarly, first and second signals are associated with a second encoded particle set containing human DNA from chromosome 18. The first signal and the second signal associated with the second encoded particle set are compared, yielding information about the chromosome 18 DNA of the individual subject compared to the chromosome 18 reference DNA.

The figures and descriptions herein illustrate the best mode but many alternative materials and processes can be substituted. One of skill in the art will recognize appropriate alternative materials and processes and will be able to make and use the compositions and methods described without undue experimentation.

The compositions of the various buffers and other assay components may be substituted.

The conditions for culturing, purification amplification, denaturation, coupling, hybridization, reporter binding, washing, and bead handling can all be varied by the user to suit particular types of cells, template genomic DNA, samples, selected reporters and the like.

The assay in examples herein performs well with as little as 30 ng of sample DNA. In situations where the biological source yields insufficient DNA for the described assay the sample can be amplified by a variety of whole-genome amplification (WGA) methods, such as DOP PCR or phi-29 PCR. When utilizing WGA-processed samples, the reference DNA can be processed by the same method so that any sequence-specific amplification bias will be largely corrected by the sample/reference ratio of signals.

Kits for assaying DNA are provided. In particular embodiments, a kit is provided which includes an encoded particle set and/or a mixture of two or more encoded particle sets. Instructional material for use of the encoded particle set and/or multiplex reagent including two or more encoded particle sets is optionally included in a kit. An ancillary reagent such as buffers, enzymes, washing solutions, hybridization solutions, detectable labels, detection reagents and the like are also optionally included.

Embodiments of assay compositions and methods are illustrated in the following examples. These examples are provided for illustrative purposes and are not considered limitations on the scope of compositions and methods.

### EXAMPLES

### Example 1

### Preparation of a Bead Set Reagent for Genomic DNA Assay

FIG. 1A shows a flowchart illustrating preparation of BAC amplicons from a single BAC clone and immobilizing the amplicons as probes onto a set of encoded beads. In this example, the beads in the set all have the same ID code.

The starting material is living BAC clone material, 10, a long (100-200 kilobases typically) human DNA sequence inserted into the genome of an E. coli bacteria cell. A small chip of frozen BAC glycerol stock material is picked and used as the starting material for a standard bacterial cell culture process, 11. The cells are cultured in 35 ml medium in 50 ml tubes overnight at 37° C. with a selective antibiotic according to a standard BAC culture protocol. The cultured cells are then centrifuged to the bottom of the tube at 4° C. for 20 minutes and the supernatant withdrawn and discarded. The cell pellet is resuspended in a buffer containing RNase, and then lysed using LyseBlue (Qiagen, Valencia Calif.) and SDS. The lysate, 12, is centrifuged, 13, at approximately 20,000 g for 30 minutes, and the supernatant, containing the DNA in solution, is collected and the pellet discarded. The centrifugation is repeated for 15 minutes on the supernatant. The clear supernatant containing the dissolved BAC DNA is collected, while the cellular debris, proteins and other impurities are driven to the bottom of the tube and discarded. The BAC DNA is extracted and purified, 17, from the supernatant using a Qiagen Genomic-Tip 20/G column purification kit. This kit comprises purification columns, 15, and wash and elution buffers, 16. After elution, the now highly purified BAC DNA is precipitated and into pellets by isopropanol, 19, precipitation. The yield is typically 20 to 200 ng of purified BAC DNA, 18. This BAC DNA can be stored as a dried pellet or resuspended in water for use immediately in the next steps.

A quantity of PCR amplicons representing substantially the entire sequence content of each BAC DNA is then produced using two rounds of polymerase chain reaction (PCR) amplification. The first round of PCR, 20, is non-specific degenerate oligonucleotide primer (DOP) PCR using a DOP primer mix, 21, a DOP PCR polymerase, 22, and DOP PCR buffer, 23, with the above prepared BAC DNA, 18, used as template. The second round of PCR amplification, 25, utilized a single primer directed at the known sequence motifs of the DOP primers. Two rounds of PCR are used to generate yields of approximately 20 μg of final amplicon product, 29, for subsequently coupling, 32, the amplicons, 29, to encoded beads, 30.

The amplicons are prepared as follows.

A first 50 μl DOP PCR mix is made for each BAC DNA comprised of:

| | | |
|---|---|---|
| 10X DOP PCR Buffer | 5.0 | μl |
| 10 mM dNTP's (each) | 1.0 | μl |
| 50 mM MgCl | 5.0 | μl |
| 10 uM DOP Primer Mix (each) | 10.0 | μl |
| 20 to 50 ng BAC DNA Template | 2.0 | μl |
| Platinum Taq polymerase | 0.5 | μl |
| Water | 21.5 | μl |
| Total Volume | 50.0 | μl |

The DOP PCR buffer, 23, included 20 mM Tris HCL (pH 8.4), 50 mM KCl and 5 mM MgCl. The dNTPs (Amersham Biosciences, Piscataway N.J.) are at a concentration of 200 μM. The platinum TAQ polymerase (Applied BioSystems) is at a concentration of 5 units/μl. The DOP primer mix, 21, see Fiegler et al. 2003, Genes Chromosomes Cancer, 36(4):361-74, included three sets of degenerate oligonucleotides of the following 22-mer sequences (Operon Biotechnologies, Huntsville Ala.), wherein the Ns represent randomized nucleotides:

| | |
|---|---|
| 5′ CCGACTCGAGNNNNNNCTAGAA 3′ | SEQ ID No. 1 |
| 5′ CCGACTCGAGNNNNNNTAGGAG 3′ | SEQ ID No. 2 |
| 5′ CCGACTCGAGNNNNNNTTCTAG 3′ | SEQ ID No. 3 |

wherein N denotes random nucleotides.

The BAC DNA template, 18, dissolved in water, is purified by column purification, 17, using Qiagen Genomic-Tip 20/G column purification kit. The Platinum Taq polymerase, 22 (Invitrogen, Carlsbad Calif.) is at a concentration of 5 units/μl.

The first-round amplification, 20, is performed in a GeneAmp 9700 themocycler (Applied BioSystems, Foster City Calif.) according to the following temperature/time profile:

| | | |
|---|---|---|
| 3.0 min | 94° C. | |
| 1.5 min | 94° C. | |
| 2.5 min | 30° C. | 9 Cycles |
| 0.10 C./sec | 72° C. (ramp) | |
| 3.0 min | 72° C. | |
| 1.0 min | 94° C. | |
| 1.5 min | 62° C. | 30 Cycles |
| 2.0 min | 72° C. | |
| 8.0 min | 72° C. | |
| 4.0° C. | | (steady state) |

The amplicon products, 24, from this first round of DOP PCR, 20, are then used as the templates for a second round of PCR, 25. The single primer, 76, in the second round is specific to the common sequence portions of the DOP primers, 21 used in the first round, 20. This primer, 26, is amine-modified so that the resulting amplicons, 29, would also have an amine group on one end to facilitate simple coupling to the encoded beads in a subsequent step, 32.

The second round PCR is performed as follows.

A second 100 μl PCR mix is made for each BAC amplicon template including:

| | | |
|---|---|---|
| 10X PCR Buffer | 10.0 | μl |
| 10 mM dNTP's (each) | 2.0 | μl |
| 50 mM MgCl | 10.0 | μl |
| 10 uM Amine Primer | 15.0 | μl |
| Template (from PCR #1) | 2.0 | μl |
| Platinum Taq | 0.5 | μl |
| Water | 58.5 | μl |
| Total Volume | 100.0 | μl |

The PCR 2 buffer, 28, included 20 mM Tris HCL (pH 8.4), 50 mM KCl and 5 mM MgCl. The dNTPs (Amersham Biosciences, Piscataway N.J.) are at a concentration of 200 μM. The platinum TAQ polymerase (Applied BioSystems) is at a concentration of 5 units/μl.

The amine-linked primer (Operon) had the following sequence.

5′-GGAAACAGCCCGACTCGAG-3′ SEQ ID No. 4

The templates in reaction, 25, are the DOP amplicons, 24, from the previous DOP PCR round, 20. The second-round amplification, 25, is performed in a GeneAmp 9700 themocycler (Applied BioSystems) according to the following temperature/time profile:

| | | |
|---|---|---|
| 10 min | 95° C. | |
| 1.0 min | 95° C. | |
| 1.5 min | 60° C. | 35 Cycles |
| 7.0 min | 72° C. | |
| 10 min | 72° C. | |
| 4.0° C. | | (steady state) |

This second PCR product, 29, is then purified using a magnetic-bead based kit, 9, (PCR Clean Beads, Agencourt Bioscience Corp, Beverley Mass.) according to the manufacturer's protocol. The purified amplicons, 29, are then resuspended in 40 μl water and stored at −20° C. until used in the bead coupling step as described below.

The encoded bead coupling process, 32, to immobilize the amplicon product, 29, as probe DNA onto the surface of encoded beads is performed on Luminex carboxy beads, 30 (Luminex, Austin Tex.) at a scale of 50 μl of the standard bead concentration, yielding approximately 650,000 beads. The beads are made of polystyrene, approximately 5.6 μm in diameter, and encoded with controlled amounts of two or three fluorescent dyes to facilitate their bead ID being detected in a purpose-built flow cytometer reading instrument. 50 μl of suspended beads, 30, all of one bead ID or region, are transferred from the Luminex tube in which they are delivered to a 1.5 ml Eppendorf tube for the coupling, 32, with vortexing and sonication used to ensure suspension. The beads are then spun down at 12,000 RPM for 3 minutes and the bead buffer supernatant removed without disturbing the bead pellet. 25 μL of MES buffer is added to each tube of beads, followed by vortexing and sonication. Separately, 10 μg of PCR 2 amplicons, 29, from each BAC are then added to a second set of 1.5 ml centrifuge tubes, and the DNA in each tube is then dried down completely in a SpeedVac (ThermoFisher Scientific, Waltham Mass.). One bead suspension is then transferred into each DNA tube, vortexed and sonicated for 5 seconds each to mix, keeping careful track of the bead ID (region) associated with each BAC.

Next, 1.5 μl of freshly dissolved EDC, 31, (1-ethyl-3-[dimethylaminopropyl]-carbodiimide hypocloride, Pierce, Rockford Ill.) at 10 mg/ml is added to each tube, vortexed immediately, and incubated for 30 minutes at room temperature in the dark (to preserve the Luminex beads' fluorescent encoding). Remixing is performed at the 15-minute point. The EDC addition, incubation, and remixing is then repeated for a second time.

500 μl of TNT buffer (0.1M Tris pH 7.5, 0.15M NaCl, 0.02% Tween 20) is then added to each tube and vortexed. The tubes are then spun on a microfuge for 4 minutes at 12,000 RPM to drive the beads to the bottom and the supernatant carefully removed. Next, 500 μl of 0.1% SDS is added, and the beads again spun down for 4 minutes at 12,000 RPM and the supernatant carefully removed. Finally, 50 μl of 1×TE buffer (10 mM Tris pH 7.5, 1 mM EDTA) to each tube and vortexed.

The bead set, 33, with immobilized amplicon probes, 29, can be included as a component of a multiplex bead set for use in assays of genomic DNA.

### Example 2

### Preparation of a Multiplex Encoded Bead Set Reagent for DNA Assay

FIG. 2 is a flowchart illustrating mixing m different encoded bead sets, each with its respective immobilized BAC-amplicon probe DNA, together to make a multiplexed encoded bead set.

Encoded bead sets 34, 35, 36, and 37 are forced into suspension by sonication, rotation of a tube container, vortexing or a similar method. A pipette is then used to transfer aliquots of each bead set into another vessel where the individual bead sets are combined and mixed, followed by denaturation, 38, to facilitate subsequent hybridization to the probe DNA immobilized on the beads in an assay.

In a detailed example, the 50 μl contents of 2 or more bead sets, each in an individual tube, each encoded bead set with immobilized probe DNA, 33, are combined in batches into one 1.5 ml centrifuge tube. After combining approximately 10 bead sets, the tube is spun down and the supernatant carefully removed, in order to keep the volume down. This is repeated until all of the bead sets are combined (up to 100 encoded bead IDs or regions are supported by the Luminex 200 system, for example).

After all of the bead sets are combined into a multiplex bead set the immobilized probe DNA is denatured. After spinning down the beads and removing the supernatant, 500 μl 0.1N NaOH is added and allowed to incubate for 2 minutes at room temperature. The beads are then spun down and the supernatant carefully removed. 500 μl of 10 mM Tris, 15 mM NaCL, 0.2% Tween 20 is added, the tube vortexed, then the beads spun down and the supernatant removed. This wash step is then repeated. Finally, the volume is brought to 500 μl with 1×TE buffer, and the multiplex bead set, 39, stored in the dark at 4° C. until used for an assay.

### Example 3

### Multiplexed Genomic Gain and Loss Assay

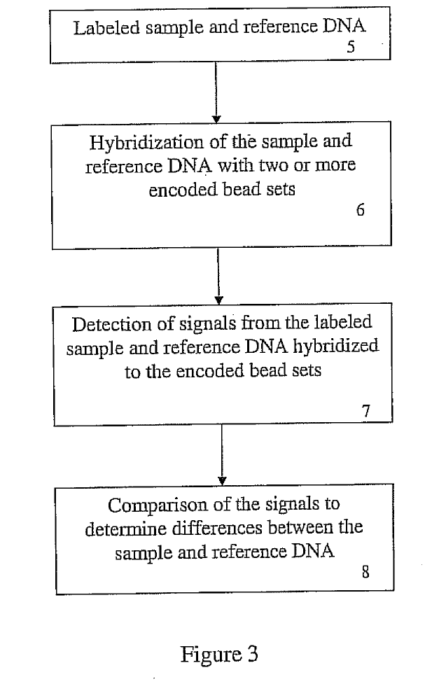

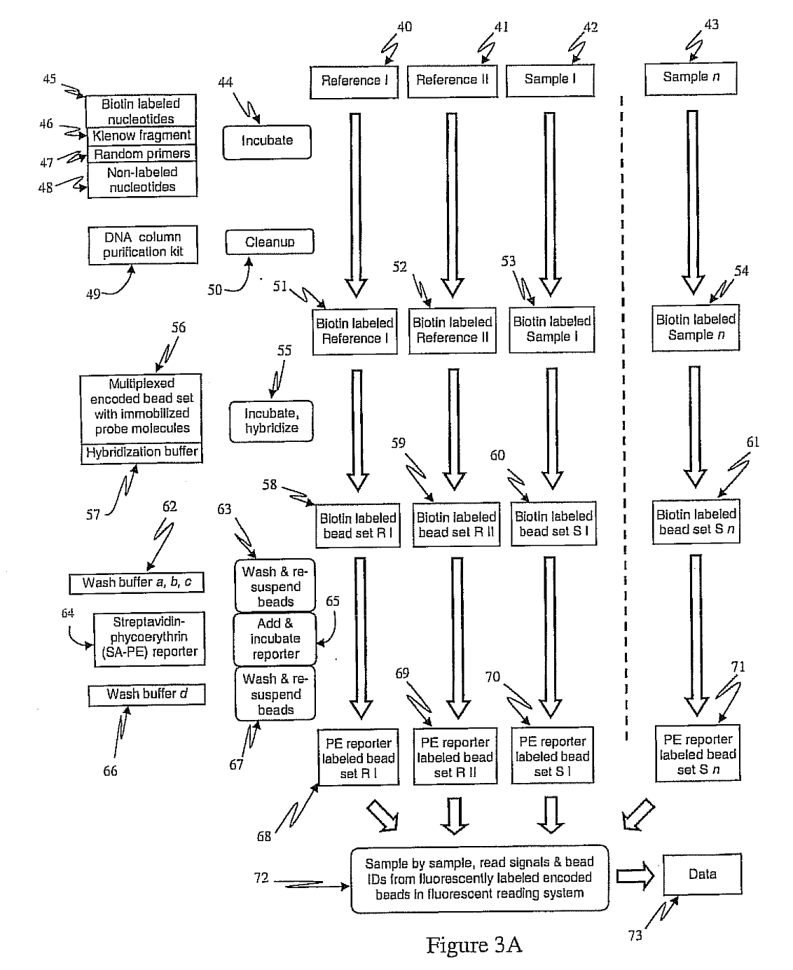

FIG. 3 is a flowchart illustrating an embodiment including running a multiplexed genomic gain and loss assay on n samples using a multiplexed encoded bead set. The flowchart shows embodiments of methods including providing labeled sample and reference DNA, 5, hybridization of the sample and reference DNA with two or more encoded bead sets, 6, detection of signals from the labeled sample and reference DNA hybridized to the encoded bead sets, 7, and comparison of the signals to determine differences between the sample and reference DNA, 8.

FIG. 3A is a flowchart illustrating an embodiment including running a multiplexed genomic gain & loss assay on n samples using a multiplexed encoded bead set.

In this example, two DNA samples and two references are being assayed in parallel. In practice, several dozen samples may be run simultaneously in parallel in a microplate format. More or fewer samples and references than this number can be assayed in parallel.

In this example, the four DNA samples, 40 and 41 representing two references and 42 and 43 representing two assay samples, are enzymatically labeled with biotin and purified. Reference samples are typically normal male and female pooled samples, such as Human Female Genomic DNA and Human Male Genomic DNA (Promega, Madison Wis.). Each DNA sample and reference is combined with biotin-labeled nucleotides, 45, (PerkinElmer, Boston Mass.), non-labeled nucleotides 49, (PerkinElmer), random primers, 47, (Operon, Biotechnologies, Huntsville Ala.) and a Klenow fragment polymerase enzyme, 46 (Epicentre Biotechnologies, Madison Wis.). After incubation, 44, the reaction product is cleaned up, 50, using a DNA column purification kit, 49, such as a Purelink DNA Mini Kit (Invitrogen). Approximately 5 μl at approximately 200 ng/μl of labeled sample is used for subsequent hybridization in the assay.

Each biotin-labeled sample or reference, 51-54, is then hybridized, 55, with the probes immobilized on the beads of a multiplexed encoded bead set, 56. Approximately 500 beads from each bead set (each probe type) are used; in this 55-plex example a total of about 55×500=27,500 beads per hybridization is used.

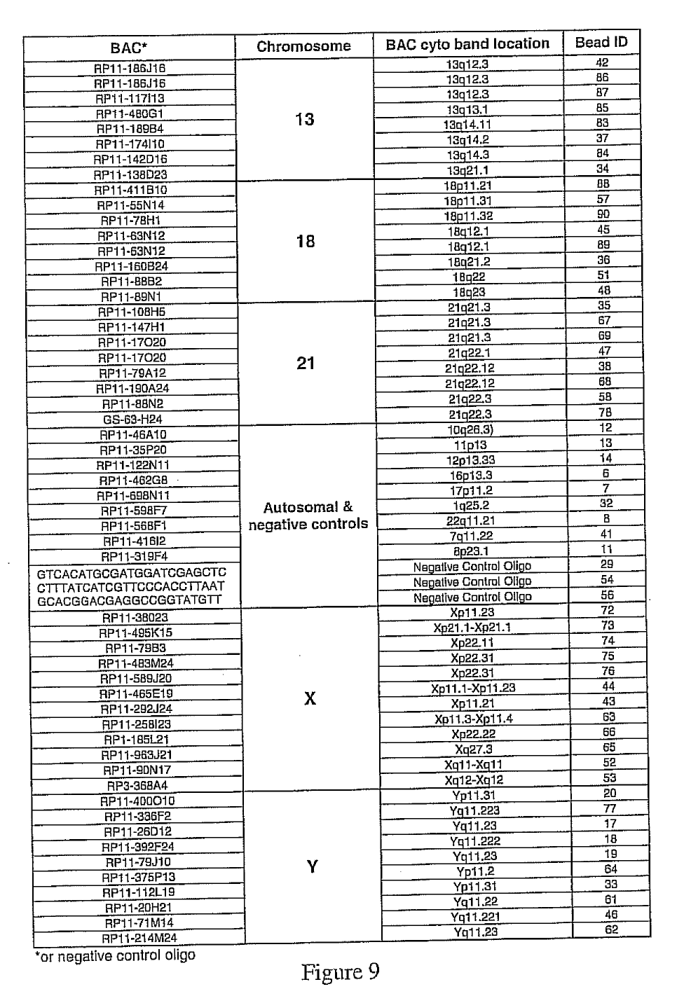

Beads of each encoded bead set are distinguishable from beads of each of the other encoded bead sets due to the encoding. Each of the 55 bead sets includes a plurality of encoded beads having attached amplicons representing substantially an entire template genomic DNA fragment. The template DNA for each bead set represents a genomic locus listed in FIG. 9.

A hybridization buffer containing Cot-1 DNA, formamide, dextran sulfate and 1.9×SSC is included in the hybridization reaction. The total volume is approximately 15 μl and the reactions are carried out in the wells of a rigid PCR-type microplate, such as the Bio-Rad HSP 9631 (Bio-Rad Laboratories, Hercules Calif.). The plate is sealed tightly to minimize evaporation using an aluminum foil sealer (MSF 1001, Bio-Rad). The hybridization incubation, 55, is performed overnight at 50° C. in a microplate shaking incubator at 1150 rpm (Wallac NCS Incubator, PerkinElmer).

After the hybridization incubation, 55, the four multiplex bead sets hybridized to the four samples, 58-61, are ready for a hybridization wash, 53, followed by incubation with a fluorescent reporter, 65, and a reporter wash, 67. First, 100 μl wash buffer a (2×SSC, 50% formamide) is added to each well, the plate resealed and incubated in the shaking incubator with 1150 rpm agitation at 50° C. for 20 minutes. The contents of each well is then transferred to a Millipore 0.46 μm HT filter plate (Millipore, Billerica Mass.). The liquid is then removed from each well by vacuum using a Millipore MSVMHTS00 vacuum manifold. Next, 100 μl of wash buffer b (2×SSC, 0.1% Igepal detergent) is added to each well, followed by another 20 minute 50° C. shaking incubation and vacuum aspiration. Then, 100 μl of wash buffer c (0.2×SSC) is added to each well and the 20 minute 50° C. shaking incubation is repeated, followed by vacuum aspiration.

100 μl of 1× PhycoLink SA solution, the streptavidin-phycoerythrin reporter, 64, is then added to each well. This reporter solution is made from 2 μl 500× PhycoLink SA PJ13S (Prozyme, San Leandro Calif.) mixed into 1 ml of reporter diluent, where the diluent is 1×PBS, 0.1% BSA and 0.05% Tween 20. This reporter solution is incubated with the multiplex bead sets for 30 minutes at 25° C. and 1050 RPM in the shaking incubator. After incubation, the solution is aspirated from the wells of the filter plate using the vacuum manifold as in the previous wash steps.

The beads are then washed twice, 67, with wash buffer d, 66, which is 1×PBS with 0.01% Tween 20. 100 μl is added to each well of the filter plate, then the liquid is vacuum aspirated through the filters in the bottoms of the plate wells. 100 μl is added a second time and incubated in the shaking incubator for 2 minutes at 25° C. at 1050 RPM. This second wash is not aspirated but used to suspend the beads for reading.

The four bead sets in the example, 68-71, are then ready to read, 72, on a Luminex 200 system (Luminex Corporation, Austin Tex.). The signals and bead IDs from the beads in each well are read in sequence, and the median fluorescence intensity of the first 50 beads of each bead ID (bead region) is recorded for each well or sample, and output in a data file, 73. There is no evidence of bead networking; the Luminex reader is set to analyze 50 beads of each region and no failures are recorded.

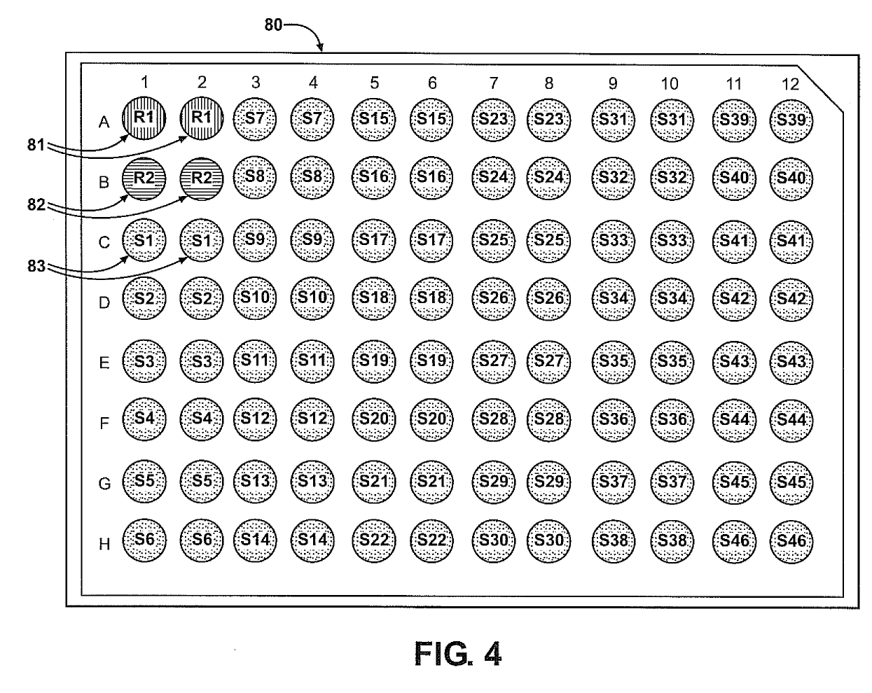

FIG. 4 is a schematic diagram of a 96-well SBS-standard microplate, 80, showing example locations of duplicate references and duplicate samples for running the assay on 46 samples in parallel. Duplicate hybridizations of each labeled sample can be used to assure data generation in case of a well-sealing failure that results in evaporation of the reagents from a single well. When the duplicate is not affected data is still generated from that sample. Using this microplate and encoded bead approach a single laboratory technician can assay, for example, 46 samples and 2 references at a time, all in duplicate, labeling on a first day, hybridizing overnight, and washing & reading on the second day. The assay can alternately be run without replicates or with more than two replicates. Shown are duplicates of two references, 81 and 82, and duplicates of samples, and example of which is indicated at 83.

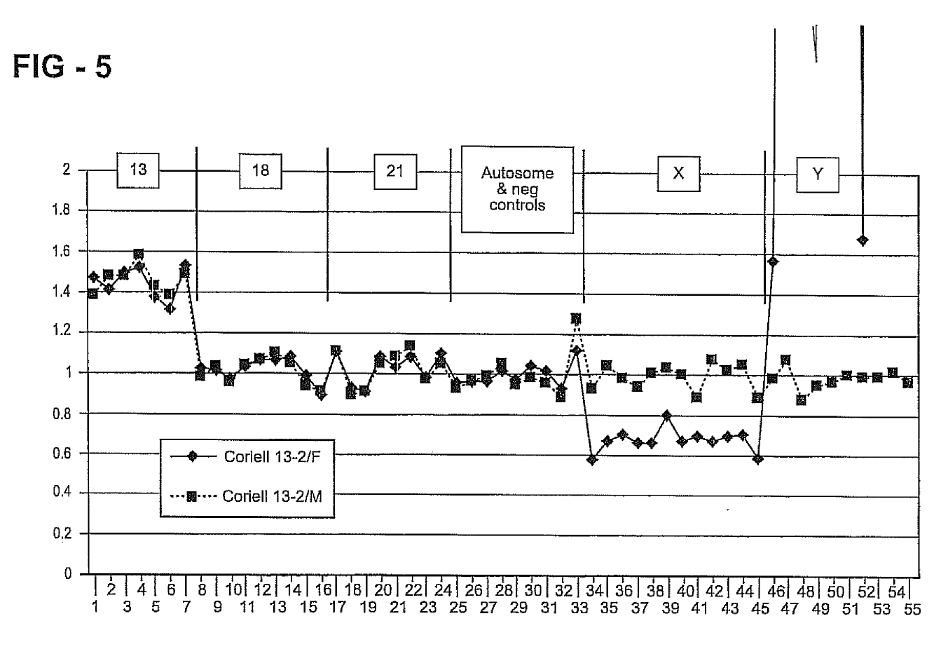

FIG. 5 is an example of data generated using a Coriell DNA sample having a trisomy on chromosome 13, sex male;

This data is calculated from the median fluorescence values for each bead region produced by the Luminex reader. The average values of the negative control beads 29, 54, and 56 are subtracted from all other signals (see FIG. 9). The signals from nine autosomal clones are then ratioed with the corresponding clone signals from the male and female reference DNAs. A normalization factor is calculated such that when the factor is applied to all of the autosomal clone signals it drove the average autosomal ratio to a value of one. This normalization factor is then applied to all of the signals for the sample.

The resulting ratios are plotted and shown in FIG. 5. Note that the ratios for the chromosome 13 clones are all in the range of 1.3 to 1.6, while the clones for chromosomes 18 and 21, as well as the other autosomal clones are but one all below 1.2. The trisomy in chromosome 13 is readily apparent. Also, the ratio plot of the sample compared to male reference (square data points) is effectively flat across the X and Y sex chromosome. This is the response expected from a male sample. The plot of the sample compared to female reference (diamond data points) shifts down for X and up for Y, also as expected for a male sample.

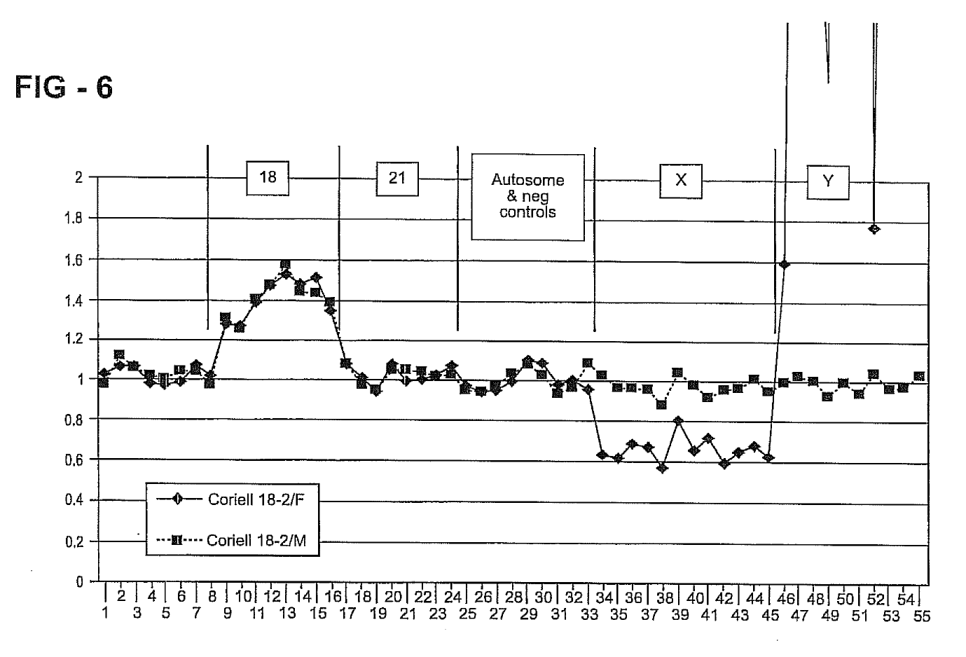

FIG. 6 is an example of data generated using a Coriell DNA sample having a trisomy on chromosome 18, sex male. The data is generated and plotted as described for FIG. 5.

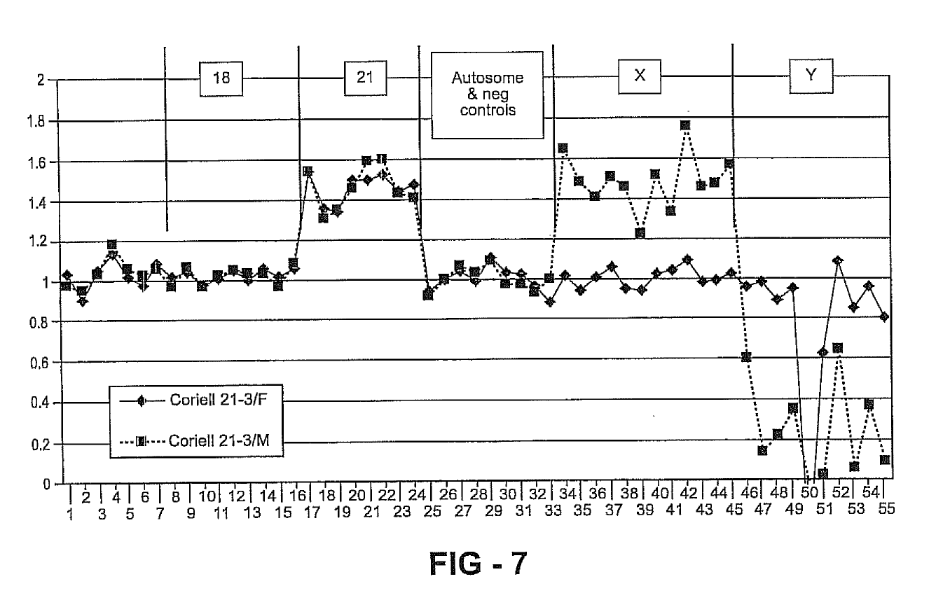

FIG. 7 is an example of data generated using a Coriell DNA sample having a trisomy on chromosome 21, sex female. The data is generated and plotted as described for FIG. 5.

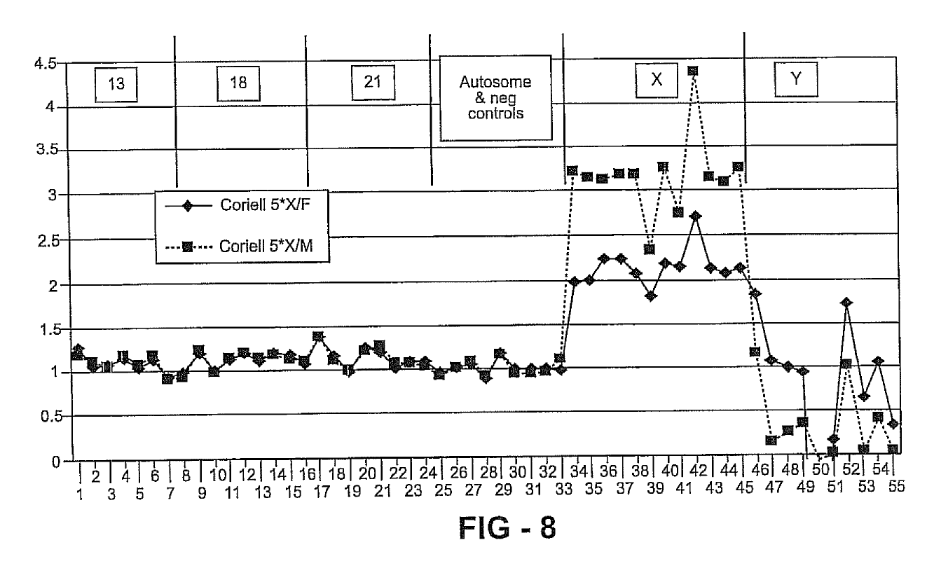

FIG. 8 is an example of data generated using a Coriell DNA sample having a 5-copy amplification of the X chromosome. The data is generated and plotted as described for FIG. 5.

FIG. 9 is a table displaying the BAC clones having human genomic DNA inserts used to generate amplicons in the example assays, their chromosome and cytoband locations, the sequence of the negative control oligonucleotides, and the bead ID (Luminex bead region) for the bead set to which each amplicon probe is immobilized. Sequentially numbered plotted points on the x-axis in FIGS. 5-8 are associated with BACs listed top-to-bottom in FIG. 9. BAC RP11-186J16 is immobilized to two different bead regions (42 and 86).

For a negative control, an oligonucleotide that has no sequence homology to the human genome is selected. Specific negative control oligonucleotides used are

| | |
|---|---|
| 5′ GTCACATGCGATGGATCGAGCTC 3′ | SEQ ID No. 5 |
| 5′ CTTTATCATCGTTCCCACCTTAAT 3′ | SEQ ID No. 6 |
| 5′ GCACGGACGAGGCCGGTATGTT 3′ | SEQ ID No. 7 |

The signals generated by the three bead regions 29, 54, and 56 having attached negative control oligonucleotides are averaged and subtracted from all other bead signals prior to calculating ratios.

### Example 4

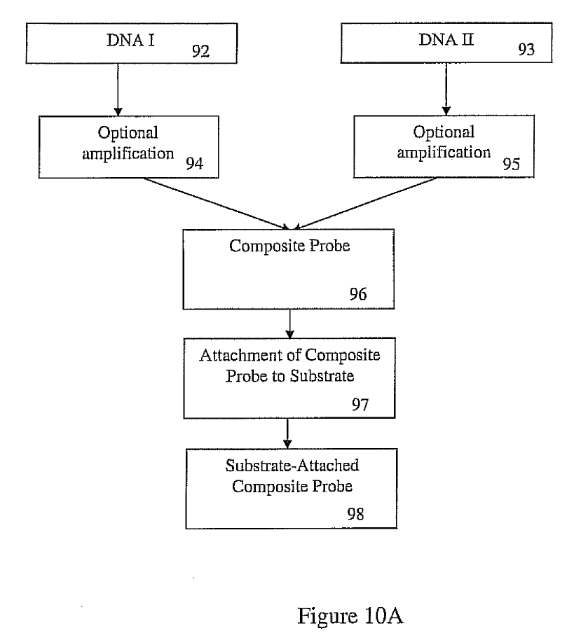

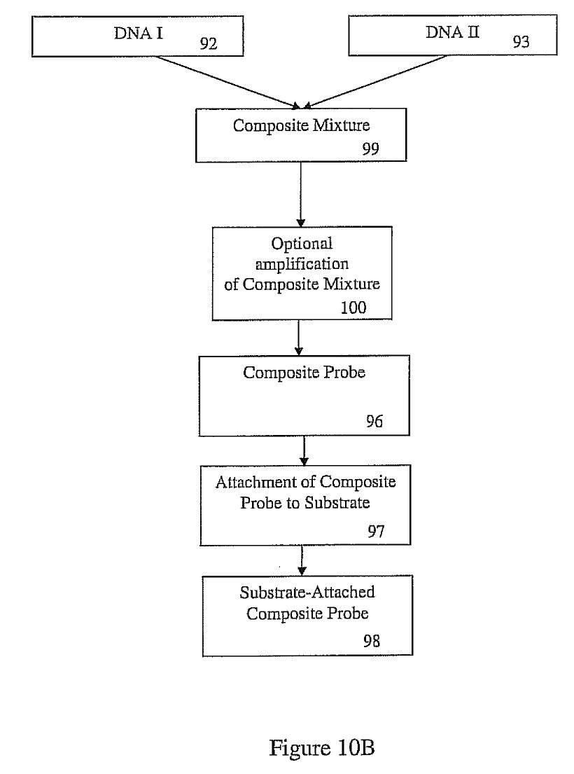

FIG. 10A is a schematic flowchart illustrating a process for making a composite probe according to one aspect described herein. Probe DNA, 92, from one source and probe DNA, 93, from a second source are optionally amplified by PCR separately, 94 and 95, to produce amplicon probes which are then mixed to form a composite probe, 96. The composite probe is attached, 97, to a substrate to form a substrate-attached composite probe, 98

FIG. 10B is a schematic flowchart illustrating a process for making a composite probe according to one aspect described herein in which the probe DNA, 92 and 93, can be pooled to form a composite mixture 99, prior to optional PCR amplification 100, to produce the composite probe material, 96, which is attached is attached, 97, to a substrate to form a substrate-attached composite probe, 98.

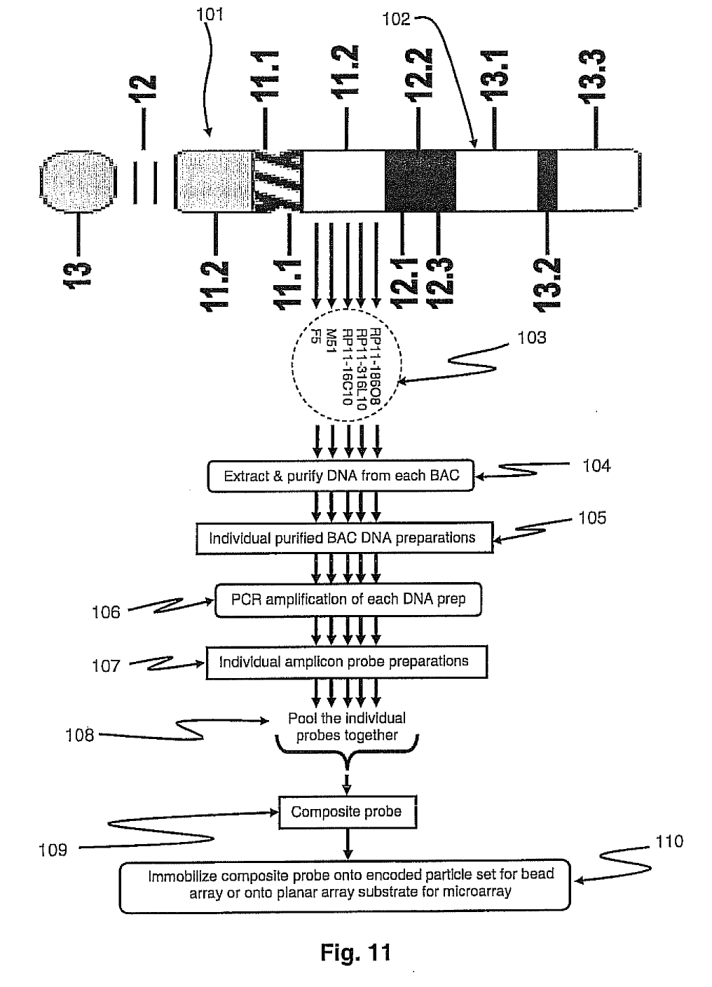

FIG. 11 is a schematic flowchart showing a process for making composite probes according to one aspect of a process described herein. An ideogram, 101, showing the cytobands, 102, of the chromosome of interest (chromosome 22 in this case) is shown. A set of five BACs, 103, with genome loci in the region of interest for the assay is shown with each BAC's genome locus approximately placed on the ideogram. In this case, DNA from five BACs mapping to cytoband 22p11.2 corresponding to the DiGeorge microdeletion syndrome were used to make the composite probe. The ideogram in this figure is schematic to show the genomic proximity of the five example BACs selected from one cytoband; the BAC DNA is not extracted from a human chromosome in this process.

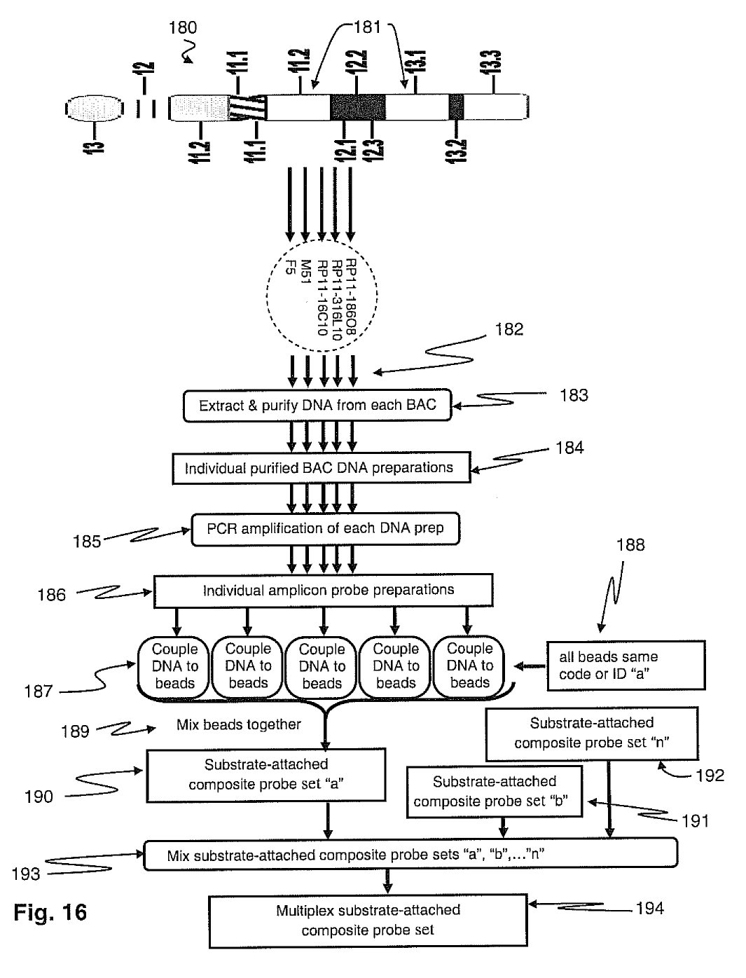

DNA was extracted and purified from each of the five cultured BACs utilizing conventional protocols. The cultured bacterial cells were lysed, the DNA was precipitated and then purified, 104, using centrifugation and column purification (Qiagen, Valencia Calif.). The purified DNA from each BAC, 105, was then used as the template for degenerate oligonucleotide primer (DOP) PCR amplification, 106. This was in turn followed by specific PCR amplification of the DOP product using the DOP primer sequence as the specific PCR primer. This process produced five individual amplicon probes, 107. These individual probes, 107, were next pooled together, 108, to produce a composite probe, 109. The composite probe was then immobilized, 110, to a set of Luminex encoded multiplex microspheres, all of one bead “region”; i.e. having the same bead encoding identification, for use as an 22p11.2 cytoband probe in a multiplex genomic gain-loss assay. The individual probes, 107, were each also immobilized individually, each to a bead set with a unique identifier so that their responses could be compared to that of the composite probe. Another protocol for preparing such probes is exemplified in FIG. 16.

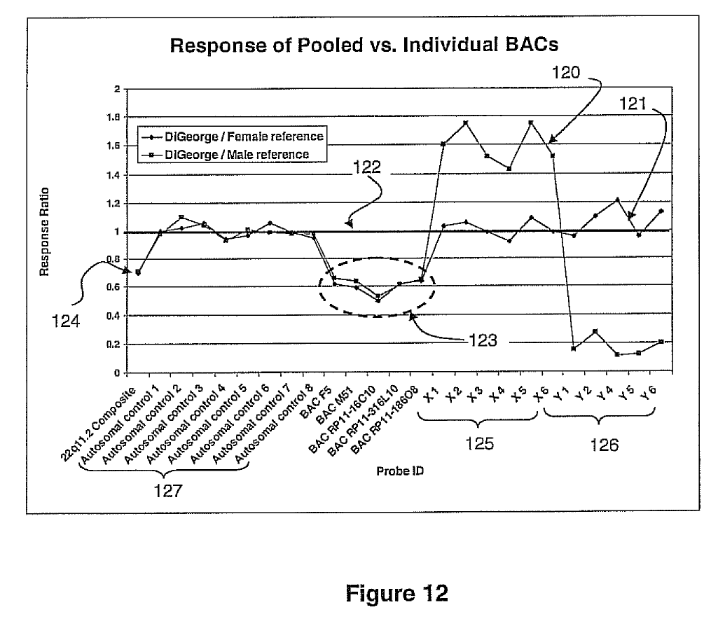

FIG. 12 is a data plot from a test assay demonstrating the use of a composite probe with a DiGeorge syndrome reference DNA sample (Coriell Institute for Medical Research, Camden N.J.). The assay was performed on the Luminex xMAP platform using immobilized PCR-product probes on the Luminex encoded microspheres. The PCR product probes were made using DOP PCR from BAC DNA. The probes were immobilized, each probe on a microsphere set separately identifiable by the Luminex system, and a test assay run as described herein. The multiplex probe panel included eight autosomal probes from genome loci not expected to show a gain or loss between the DNA sample and the male and female DNA references. It also included six X chromosome probes and five Y chromosome probes as positive controls, so that when the test sample is compared to a reference of the opposite sex the ratio response of a known gain or loss in the sex chromosomes can be observed. The panel also included five 22q11.2 probes, 123, see Table I below, at the locus of the DiGeorge syndrome deletion. The center loci of these five BACs span about 0.45 megabases (445 kilobases) and the total span accounting for their 175 kb typical length is a little over 600 kilobases.

TABLE I
BACs and the Chr 22 linear mapping locations
of their centers (megabases)

| BACS | pter |
|---|---|
| F5 | 17.7765040 |
| M51 | 17.8000000 |
| RP11-16C10 | 17.9469780 |
| RP11-316L10 | 18.1740250 |
| RP11-186O8 | 18.2284550 |
| | qter |

Referring to FIG. 12, there are two data series in the ratio plot of the DiGeorge syndrome sample compared to both male and female normal reference DNA. Data series 120 is the ratio response of the DiGeorge sample compared to male reference DNA; it shows a relative gain at all of the X chromosome probes, 125, and a loss at all of the Y chromosome probes, 126. This response shows that the sample is from a female. Data series 121 is the same sample compared to female reference DNA, and it shows a ratio response near 1.0 across the X and Y sex chromosome probes, confirming that the sample is from a female. The 1.0 ratio line, 122, in the plot indicates the expected result of sample/reference ratio for any given probe where the sample has no genomic gains or losses compared to the normal references. The autosomal probes, 127, were put into this panel as controls expected to produce a ratio of approximately 1.0, which they did.

Five probes, 123, from the 22q. 11.2 locus were included in the panel. These five probes all show a ratio <1 compared to both male and female reference DNA, consistent with the known genomic deletion in the sample at that locus. Finally, one multiplex bead type was coupled to a composite probe comprising a mixture or pool of the five 22q11.2 probes, 123. The composite probe ratio response, 124, also indicating a deletion, is reasonably concordant with the average of the response of the five constituent probes that were pooled.

### Example 5

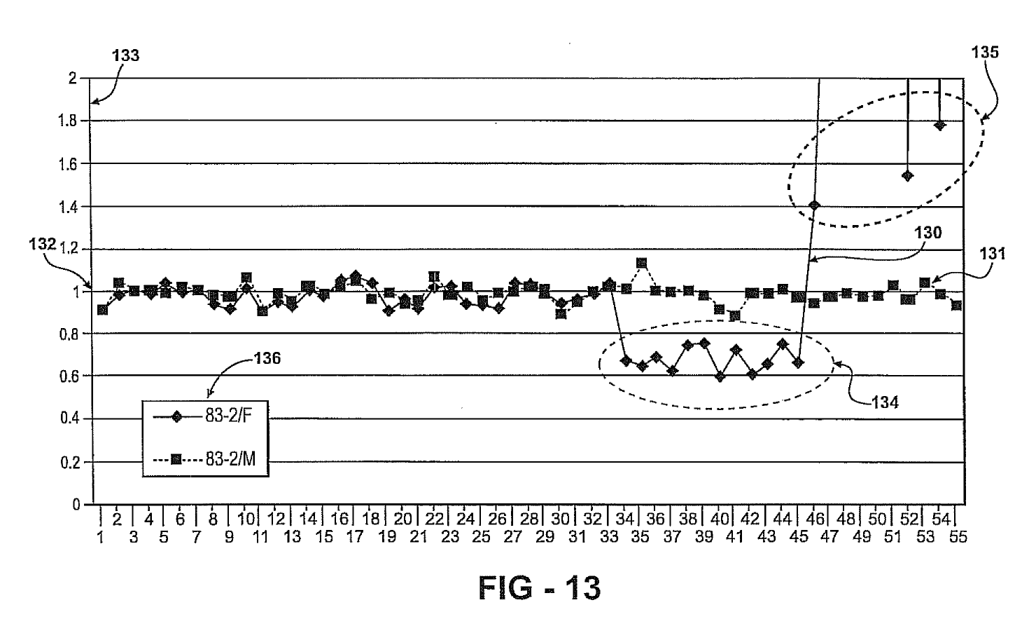

FIG. 13 is ratio data from a Luminex bead array gain-loss assay according to one aspect of the present invention. This data is from fetal DNA extracted from a prenatal amniotic fluid sample for which the fetal sex was not definitively known. The assay was run with both male and female references assayed simultaneously in different wells of the same 96-well microplate. The legend, 136, identifies the two data displays, referenced to female (diamond plot points) and referenced to male (square plot points). The horizontal ratio=1.0 line, 132, is centered in the plot. The ratio scale, 133, is the vertical axis of the graph. A first data plot, 130, is shown with the ratio of sample/reference generated against a female reference. For this plot the data for X chromosome probes, 134, shows a ratio <1 and the data for the Y chromosome probes, 135, shows a gain compared to female. This is consistent with a male sample. The second data plot, 131, utilizing the male reference clusters closely to the ratio=1.0 line, also consistent with a male sample. Both data sets are show no significant deflection for the other probes in the array to the left of the X probes, indicating a normal sample.

Table II shows BAC identity associated with sequentially numbered plotted points on the x-axis in FIGS. 13 and 14.

TABLE II

| | CytoBand Location | Clone ID |
|---|---|---|
| 1 | 13q12.3-13q14.13 | RP11-117I13 |
| 2 | 13q12.3-13q14.3 | RP-11-186J16 |
| 3 | 13q12.3-13q14.3 | RP-11-186J16 |
| 4 | 13q13.1-13q14.3 | RP11-480G1 |
| 5 | 13q14.11-13q14.3 | RP11-189B4 |
| 6 | 13q14.2 | RP-11-174I10 |
| 7 | 13q14.3-13q21.31 | RP11-142D16 |
| 8 | 13q21.1-13q21.33 | RP-11-138D23 |
| 9 | 18p11.21 | RP11-411B10 |
| 10 | 18p11.31 | RP11-55N14 |
| 11 | 18p11.32 | RP11-78H1 |
| 12 | 18q12.1 | RP-11-63N12 |
| 13 | 18q12.1 | RP-11-63N12 |
| 14 | 18q21.2 | RP-11-160B24 |
| 15 | 18q22-18q22 | RP-11-88B2 |
| 16 | 18q23 | RP11-89N1 |
| 17 | 21q21.3 | RP11-108H5 |
| 18 | 21q21.3-21q21.3 | RP-11-147H1 |
| 19 | 21q22.12 | RP11-17020 |
| 20 | 21q22.12 | RP11-17020 |
| 21 | 21q22.1-21q22.1 | RP-11-79A12 |
| 22 | 21q22.3 | GS-63-H24 |
| 23 | 21q22.3 | RP11-190A24 |
| 24 | 21q22.3 | RP11-88N2 |
| 25 | Auto 10q26.3 | RP11-462G8 |
| 26 | Auto 11p13 | RP11-698N11 |
| 27 | Auto 12p13.33 | RP11-598F7 |
| 28 | Auto 16p13.3 | RP11-568F1 |
| 29 | Auto 17p11.2 | RP11-41612 |
| 30 | Auto 1q25.2-1q31.1 | RP11-46A10 |
| 31 | Auto 7q11.22 | RP11-35P20 |
| 32 | Auto 8p23.1 | RP11-122N11 |
| 33 | Auto 22q11.21 | RP11-319F4 |
| 34 | Xp11.1-Xp11.23 | RP11-465E19 |
| 35 | Xp11.21 | RP-11-292J24 |
| 36 | Xp11.23 | RP11-38023 |
| 37 | Xp11.3-Xp11.4 | RP-11-258I23 |
| 38 | Xp11.4-Xp21 | RP11-495K15 |
| 39 | Xp22.22 | RP11-185L21 |
| 40 | Xp22.31 | RP11-79B3 |
| 41 | Xp22.31 | RP11-483M24 |
| 42 | Xp22.31 | RP11-589J20 |
| 43 | Xp27.3 | RP-11-963J21 |
| 44 | Xq11-Xq11 | RP-11-90N17 |
| 45 | Xq12-Xq12 | RP3-368A4 |
| 46 | Yp11.2 | RP-11-375P13 |
| 47 | Yp11.31 | RP11-400O10 |
| 48 | Yp11.31 | RP-11-112L19 |
| 49 | Yq11.22 | RP-11-20H21 |
| 50 | Yq11.221 | RP-11-71M14 |
| 51 | Yq11.222 | RP11-392F24 |
| 52 | Yq11.223 | RP11-336F2 |
| 53 | Yq11.23 | RP11-26D12 |
| 54 | Yq11.23 | RP11-79J10 |
| 55 | Yq11.23 | RP-11-214M24 |

FIG. 14 is ratio data from the same Luminex bead array assay utilizing a sample (Coriell Institute of Medical Research, Trenton N.J.) with previously characterized genomic aberrations; trisomy 18 and XXX. Again, two data plots are displayed simultaneously for the sample referenced to female, 145, and referenced to male, 146. The probes for trisomy 19, all produce ratio data showing a gain, 142, compared to both references. The data for the X probes shows a gain, 144, on the female-referenced plot, 145. This gain is of the same magnitude as the trisomy gain, 142, which is consistent with XXX (sample) ratioed to XX (female reference). The male-referenced plot, 146, shows a much larger gain, 143, on the X probes as would be expected with XXX (sample) ratioed to X (male reference). The probes for the Y chromosome are noisy, as is common in aCGH, but the remaining probes are all clustered closely around the ratio=1.0, line 141. The ratio scale, 140, is the vertical axis of the graph.

It is apparent from these examples that the sex of a sample with a normal complement of X and Y chromosomes is immediately apparent from the ratio data generated against both male and female references. It is also apparent in the case of a multi-X aberrant sample the quantitation of the multiple copies of X is more straightforward when using both references.

### Example 6

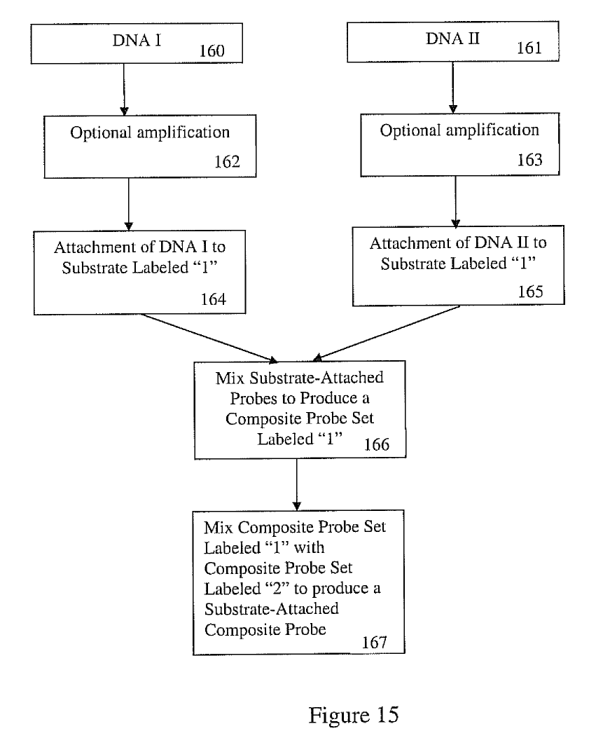

FIG. 15 is a schematic flowchart illustrating a process for making a substrate-attached composite probe according to one aspect described herein. Probe DNA I, 160, from one source, such as a BAC, and probe DNA II, 161, from a second source, such as a BAC, are optionally amplified by PCR separately, 162 and 163, to produce amplicon probes. The probe DNA I, 160, or amplicon DNA, is attached, 164, to a first labeled substrate, such as beads. The probe DNA II, 161, or amplicon DNA, is attached, 165, to a second labeled substrate, such as beads. The first and second labeled substrates have the same label such that the substrates are indistinguishable. The resulting substrate-attached DNA probes are then mixed to form a substrate-attached composite probe set, 166. Optionally, the substrate-attached composite probe set, 166, is mixed with a second substrate-attached composite probe to produce a multiplex substrate-attached composite probe set, 167. The labeled substrates of the second substrate-attached composite probe set have a different label such that the substrates of the first and second substrate-attached composite probe sets are distinguishable.

FIG. 16 is a schematic diagram of an embodiment of a process for a genomic gain/loss assay using at least one substrate-attached composite probe. Referring to FIG. 16, an ideogram, 180, showing cytobands, such as those designated 181, of a chromosome of interest, such as chromosome 22, shown here. DNA, 183, is extracted from a plurality of BAC clones 182 where the plurality of BAC clones are extracted from one or more regions of the genome or chromosome for which a single gain-loss determination is desired.

Each individual DNA preparation, 184, is separately amplified by PCR, 185, resulting in an amplicon probe preparation, 186, corresponding to each BAC. Each one of these amplicon probe preparations is separately coupled, 187, to a set of encoded beads, 188, such as Luminex carboxy beads, where all of the beads have the same coding (or ID, or “region” in Luminex terminology). The bead set coding is identified as “a” in this example. After coupling the probes to beads, the identically coded probe-coupled beads are then mixed, 189, to form a substrate-attached composite bead set “a” 190.

In some embodiments of assays, such as a multiplex bead-array assay, additional substrate-attached composite probe bead sets “b” . . . “n” (191 . . . 192) are mixed, 193, with the substrate-attached composite probe set “a” to make a multiplex substrate-attached composite probe bead set, 194. Any number “n” of additional substrate-attached composite bead sets can be mixed to make a multiplex substrate-attached composite probe set or mix.

In addition to one or more substrate-attached composite probe sets, one or more additional substrate-attached probes can be included in a multiplex probe mix. Each of the additional substrate-attached probes has its own code, ID, or “region” so that results from a multiplex assay distinguish the probe sets.

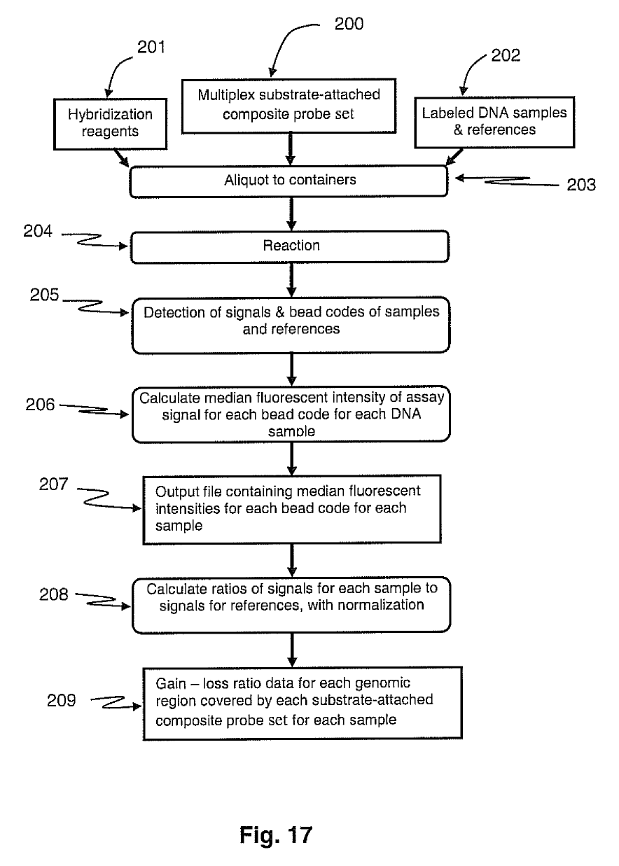

FIG. 17 is a schematic process flow chart showing an embodiment of a method using a multiplex substrate-attached composite probe mix in a multiplex genomic DNA gain-loss assay. In an embodiment of an assay process, exemplified in FIG. 17, a multiplex substrate-attached composite probe bead set, 200, hybridization reagents, 201, and labeled DNA samples and references, 202, are aliquoted to containers, such as wells of a microplate, generating assay mixtures. Exemplary reagents and reaction conditions such as described in Example 4 can be used in conjunction with the assay described in this example.

The assay mixtures are hybridized, 204, for several hours under standard hybridization conditions and then washed using appropriate wash conditions for the probes used.

In this example, a reporter is added to each container and incubated in the assay mixture for an appropriate time, followed by washing, to detect the labeled sample and reference DNA. At this point each container contains a multiplex composite bead mix where each bead carries fluorescent reporter in approximate proportion to the concentration of complementary labeled DNA for the sample in that container.

Signals from each container are detected, 205, for example, using a specialized flow-cytometer reader such as the Luminex (Austin, Tex.) 100 or 200 instruments. In this example, the fluorescent assay signals and bead code (or “region”) for each bead in each container are detected. The median fluorescent intensity (MFI) of all of the assay signals from each bead code or region in each container is calculated, 206. The median values are then reported out, 207, via a data file, with the MFI for each bead code or region for each container or sample.

Normalized ratios for each bead code for each sample are calculated, 208, along with those from the references. These normalized ratios are the genomic DNA gain-loss data, 209, for each genomic region represented by each substrate-attached composite probe bead set.

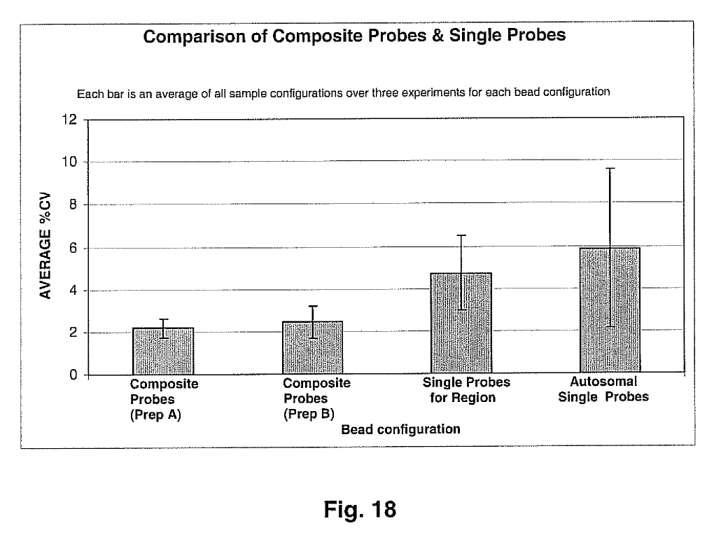

In particular embodiments described herein for assays using composite probes the signals of the individual BAC-derived probes constituting the composite probe are averaged, wherein the averaging is performed by physically mixing the probes and utilizing the resulting hybridization signal. In particular embodiments of assays described herein, medians of signals are determined. For example, the median fluorescent intensity of each bead set for each sample is determined. Median values are often less noisy than mean values since outliers of large magnitude cannot move the result as they can in a mean value calculation. Experimental data confirms this as shown in FIG. 18.

### Example 7

Three BAC clones from Chromosome 9 are used in this example illustrating both assays including a multiplex bead set or a substrate-attached composite bead set.

In addition to the probes derived from the chromosome 9 BACs, individual BAC probes from other chromosomes including X, Y, 13, 18, and 21 were added to the substrate-attached composite probe set, which was prepared using two methods. The first method is described generally in FIG. 16 (Prep A in FIG. 18) while the second method is described generally in FIG. 11 (Prep B in FIG. 18). The resulting multiplex substrate-attached composite probe bead sets were assayed in six different sample and reference combinations:

Female Reference/Male Reference

Female Reference/Male WGA

Female WGA/Male WGA

Female Reference/Chromosome 9 male trisomy

Female Reference/Chromosome 9 male trisomy

### WGA

Female WGA/Chromosome 9 male trisomy WGA

Each assay was run 3 times, and the ratio coefficient of variation (CV) was calculated for each subset of probes. Referring to FIG. 18, the multiplex substrate-attached composite probe sets result showed the lowest CV and the smallest variation in the CV.

Any patents or publications mentioned in this specification are incorporated herein by reference to the same extent as if each individual publication is specifically and individually indicated to be incorporated by reference. U.S. patent application Ser. No. 11/615,739, filed Dec. 22, 2006; U.S. patent application Ser. No. 12/055,919, filed Mar. 26, 2008; and U.S. Provisional Application Ser. Nos. 60/753,584, filed Dec. 23, 2005, 60/753,822, filed Dec. 23, 2005, 60/765,311, filed Feb. 3, 2006, 60/765,355, filed Feb. 3, 2006, and 60/992,489, filed Dec. 5, 2007, are all incorporated herein by reference in their entirety.

The compositions and methods described herein are presently representative of certain embodiments, exemplary, and not intended as limitations on the scope of the invention. Changes therein and other uses will occur to those skilled in the art. Such changes and other uses can be made without departing from the scope of the invention as set forth in the claims.

1. A method of assaying a DNA sample, comprising:
providing a substrate-attached composite nucleic acid probe, wherein the substrate-attached composite probe is prepared by a process selected from (i) pooling two or more composite probes and attaching the pooled composite probes to a substrate, and (ii) combining two or more populations of substrate-attached composite probes, wherein each composite probe comprises nucleic acid sequences which specifically hybridize to at least two genomic loci in a reference genome;
hybridizing the substrate-attached composite nucleic acid probe with sample genomic DNA;
hybridizing the substrate-attached composite nucleic acid probe with reference genomic DNA;
detecting a first signal indicating specific hybridization of the substrate-attached composite nucleic acid probe with the sample genomic DNA and a second signal indicating specific hybridization of the substrate-attached composite nucleic acid probe with the reference genomic DNA;
comparing the first signal and the second signal to detect differences between the first and second signals, the differences of the first and second signals indicative of differences between the sample DNA and the reference DNA, thereby assaying the DNA sample.

2. The method of claim 1 wherein the substrate-attached composite nucleic acid probe comprises a plurality of encoded particles.

3. The method of claim 1, further comprising:
hybridizing the substrate-attached composite nucleic acid probe with second reference genomic DNA.

4. The method of claim 1 wherein the composite probes each comprise insert DNA isolated from a first and a second large-insert DNA vector.

5. The method of claim 1, wherein the composite probes each comprise amplicons, the amplicons comprising random nucleic acid sequences together representing substantially an entire genomic locus.

6. The method of claim 5, wherein the amplicons have a length in the range of about 500-1200 nucleotides, inclusive.

7. A reagent for assay of DNA, comprising:
a first composite nucleic acid probe attached to a solid substrate, the first composite nucleic acid probe comprising nucleic acid sequences which specifically hybridize to two or more genomic loci in a genomic region of a reference genome, the genomic region characterized by a first terminus and a second terminus and having an intermediate region disposed between the first terminus and second terminus of at least 400 kilobases, wherein the first composite nucleic acid probe comprises nucleic acid sequences which specifically hybridize to substantially an entire first genomic locus comprising the first terminus and to substantially an entire second genomic locus comprising the second terminus, wherein the nucleic acid sequences which specifically hybridize to substantially an entire first genomic locus comprising the first terminus are attached to a first solid substrate and the nucleic acid sequences which specifically hybridize to substantially an entire second genomic locus comprising the second terminus are attached to a second solid substrate and wherein the first and second substrates are indistinguishably encoded with respect to each other.

8. The reagent for assay of DNA of claim 7, further comprising:
a second composite nucleic acid probe attached to a solid substrate, the second composite nucleic acid probe comprising nucleic acid sequences which specifically hybridize to two or more genomic loci in a second genomic region of a reference genome, the second genomic region characterized by a first terminus and a second terminus and having an intermediate region disposed between the first terminus and second terminus of at least 400 kilobases, wherein the second composite nucleic acid probe comprises nucleic acid sequences which specifically hybridize to substantially an entire first genomic locus comprising the first terminus of the second genomic region and to substantially an entire second genomic locus comprising the second terminus of the second genomic region, wherein the nucleic acid sequences which specifically hybridize to substantially an entire first genomic locus comprising the first terminus are attached to a third solid substrate and the nucleic acid sequences which specifically hybridize to substantially an entire second genomic locus comprising the second terminus are attached to a fourth solid substrate and wherein the third and fourth substrates are indistinguishably encoded with respect to each other and distinguishably encoded with respect to the first and second substrates.

9. The reagent of claim 8 wherein the first and second solid substrates are a first and second plurality of particles.

10. The reagent of claim 9 wherein the third and fourth solid substrates are a third and fourth plurality of particles.

11. A method of preparing a substrate-attached composite nucleic acid probe, reagent for assay of DNA, comprising:
isolating a first nucleic acid sequence which specifically hybridizes to substantially an entire first genomic locus comprising a first terminus of a genomic region of a reference genome;
isolating a second nucleic acid sequence which specifically hybridizes to substantially an entire second genomic locus comprising a second terminus of the genomic region of the reference genome;
binding the first nucleic acid sequence to a solid substrate to produce a first substrate-attached nucleic acid probe;
binding the second nucleic acid sequence to a solid substrate to produce a second substrate-attached nucleic acid probe;
mixing the first and the second substrate-attached nucleic acid probes to produce a substrate-attached nucleic acid probe reagent.

12. The method of claim 11 wherein the first and the second nucleic acid sequences comprise a functional group for reaction with the solid substrate.

13. The method of claim 12, wherein the first nucleic acid sequence is isolated from a first large-insert vector and the second nucleic acid sequence is isolated from a second large-insert vector.

14. The method of claim 10, wherein the first and the second nucleic acid sequences are amplified to produce amplicons comprising random nucleic acid sequences together representing substantially an entire genomic locus, prior to attachment to the solid substrates.

15. The method of claim 10, wherein the solid substrates are indistinguishably encoded particles.


SEQUENCE LISTING (7 sequences)

SEQ ID NO: 1 - DNA, 22 bp, Artificial Sequence
  degenerate oligonucleotide
ccgactcgagnnnnnnctagaa

SEQ ID NO: 2 - DNA, 22 bp, Artificial Sequence
  degenerate oligonucleotide
ccgactcgagnnnnnntaggag

SEQ ID NO: 3 - DNA, 22 bp, Artificial Sequence
  degenerate oligonucleotide
ccgactcgagnnnnnnttctag

SEQ ID NO: 4 - DNA, 19 bp, Artificial Sequence
  primer
ggaaacagcccgactcgag

SEQ ID NO: 5 - DNA, 23 bp, Artificial Sequence
  control oligonucleotide
gtcacatgcgatggatcgagctc

SEQ ID NO: 6 - DNA, 24 bp, Artificial Sequence
  control oligonucleotide
ctttatcatcgttcccaccttaat

SEQ ID NO: 7 - DNA, 22 bp, Artificial Sequence
  control oligonucleotide
gcacggacgaggccggtatgtt